(19) United States
(12) Patent Application Publication
(10) Pub. No.: US 2017/0216401 A1
(43) Pub. Date: Aug. 3, 2017

(54) COMBINATION FOR USE IN A METHOD OF TREATING CANCER

(71) Applicant: Piotr JACHIMCZAK, Würzburg (DE)

(72) Inventors: Piotr JACHIMCZAK, Würzburg (DE); Achim AIGNER, Leipzig (DE)

(73) Assignee: Piotr JACHIMCZAK, Würzburg (DE)

(21) Appl. No.: 15/126,687

(22) PCT Filed: Mar. 17, 2015
(86) PCT No.: PCT/EP2015/055528

§ 371 Date: Sep. 16, 2016

(30) Foreign Application Priority Data
Mar. 17, 2014 (EP) 14160262.3

(51) Int. Cl. A61K 38/20; A61K 45/06; A61K 47/10; A61K 9/00; A61K 31/519; A61K 38/19

(52) U.S. Cl. CPC A61K 38/2013; A61K 31/519; A61K 38/193; A61K 47/10; A61K 9/0019; A61K 45/06

(57) ABSTRACT

The present invention refers to a combination of a TGF-beta inhibitor selected from the group consisting of an ALK-5 inhibitor, a neutralizing pan-anti-TGF-beta antibody, a neutralizing antibody against TGF-beta-1, or/and -2, or/and -3, a neutralizing anti-TGF-beta receptor type I or/and type II or/and type III antibody or binding fragments thereof with IL-2 and GM-CSF for use in a method of treating a malignant tumor in a patient. The invention is further directed to a pharmaceutical composition comprising the combination of the present invention, wherein the combination of compounds as well as the pharmaceutical composition are administered intratumorally. The combination as well as the pharmaceutical composition further comprise optionally an inhibitor of TGF-beta synthesis.

### RELATED APPLICATIONS

This application is a National Phase of co-pending PCT/EP2015/055528 filed Mar. 17, 2015, which claims priority to EP 14160262.3 filed Mar. 17, 2014, each of which is expressly incorporated by reference herein in its entirety.

The present invention refers to a combination of at least two immune-stimulating cytokines such as IL-2 and/or GM-CSF, combined with a TGF-beta inhibitor for use in a method of treating cancer comprising real-time, intratumoral, presentation of personalized tumor-associated antigens (pTAAs) in growing tumor tissue of cancer-bearing patients (4-D Vaccine). In a further embodiment, the invention refers to a pharmaceutical composition comprising this combination and a pharmaceutically acceptable carrier.

### BACKGROUND

Cancer is one of the major causes for death in the world. Novel immune therapeutic strategies show first promising results in patients. However, the high rate of non-responding tumor cells or the cancer recurrance after treatment still remains a serious problem for the development of new effective therapies. The reason for disappointing clinical results is closely related to the Intratumoral Heterogeneity, which describe extremely accelerated phenotypic differentiation of rapidly cycling cells, due to Darwinian selection process leading to dynamic heterogeneity of Tumor Associated Antigens (TAAs) expressed by proliferating tumor cells and to the Intratumoral Immunosuppression. Although, through evolutionary development, the human immune system is capable of successfully fighting off a large number of diseases including malignant cancers, nevertheless, it is not in all cases able to efficiently combat the abnormal cells. The most important reason for this is the so-called “immune-escape phenomenon”, which involves mechanisms that help the tumor to deceive the immune system and evade immune reactions. The key factor driving “immune-escape phenomenon” is the release of the immunosuppressive messenger protein Transforming Growth Factor-beta (TGF-beta). Overexpressed and locally activated TGF beta in cancer suppresses the ability of the immune system to recognize the TAAs, indeed it also inhibits the development of an effective anti-tumor immune response leading to an undisturbed (re-) growth and clinical progression of the tumor.

### Tumor Heterogenity

Most of the current experimental models of cancer do not reflect the “in vivo” complexity of naturally occurring tumors. The vast majority of human cancers arises sporadically and is clonal in origin, and progresses through sequential accumulation of somatic mutations and/or epigenetic changes in genes, whose gain or loss of function is associated with malignant transformation.

There are two major models of cancer development and progression to metastatic disease (Marjanovic et al., 2012). The clonal evolution based on Darvinian selection theory suggests that successive mutations accumulating in a given cell generate clonal outgrowths that thrive in response to microenvironmental selection pressure, dictating the continuously changing phenotype of the growing tumor. The alternative cancer stem cell (CSC) model postulate that cancer cells with similar genetic backgrounds can be hierarchically organized according to their tumorigenic potential. Evolving evidence supports a new model of tumorigenicity, in which considerable plasticity between both non-CSC and CSC models exist and is further affected by interactions with numerous non-malignant cell types recruited in a growing tumor stromal tissue from new blood vessels (Bindea et al., 2013).

Multiregion genetic analysis of tumor biopsies provided evidence of intratumoral heterogeneity in all tumors, with spacially separated heterogeneous somatic mutations and chromosomal imbalances leading to phenotypic intratumoral diversity amongst the cancer cells themselves (Gerlinger et al., 2012). Genetic mutations underlying primary tumor formation are a major source of cell-intrinsic variability with a profound impact on cell phenotype. However, the genetic mutations are not the sole determinants of cancer heterogeneity. Multistep tumorigenesis disrupts a natural epigenetic differentiation program of a normal cell of origin, which additionally influences the phenotype of a growing malignant cell. Therefore, the cell-intrinsic features must be considered in a context-dependent manner. Cell behavior is strongly influenced by factors in the microenvironment acting to promote or inhibit the aggressive cancer phenotype.

Whilst cancer cells cannot anticipate future evolutionary events, the immune system must be managed to recognize all phenotyping changes in real-time mode and guide tumor destruction by inducing specific, host anti-tumor immune responses. In this context, understanding the key processes involved in the interplay between a rapidly growing tumor and the host's immune system is fundamental for optimizing current treatment strategies.

### Cancer Immunoediting

In a simple immune response model of cancer, increased tumor antigenicity should always lead to enhancement of immune responses against tumor cells. In the course of carcinogenesis, however, when a tumor reaches a clinical size in the order of 10⁹-10¹¹ cells, there are several mechanisms by which the tumor may have escaped the immunosurveillance (Jamal-Hanjani et al., 2013). One possibility is that the antigens presented by the tumor are not strong or prevalent enough to elicit an immune response. In this case, the tumor is not recognized as a significant threat by the immune system. Another possibility is that the antigen may be present in sufficient strength, but the immunosuppressive elements may diminish the efficacy of the immune system (Prehn et al., 1989). According to a mathematical model presented by Robertson-Teesi et al., there is an optimal antigenicity for all fast-growing tumors. If they are minimally antigenic, then there is only a small immune response; if they are highly antigenic, there may be not only a large immune response, but also a large suppressive response by overexpression of TGF-beta. In between these two scenarios, the immune response may be large enough to affect the tumor, but avoid excessive promotion of Tregs and other suppressive elements like TGF-beta (Robertson-Tessi et al., 2012).

The cancer immunoediting hypothesis, as described in short above, explains why despite years of preclinical efforts and hundreds of immunologic-based clinical studies, the ability of immunotherapies to reduce tumor growth in humans are still limited (Schreiber et al., 2011). Current therapeutic manipulations of immune checkpoints like blockade of CTLA-4 or PD-1/PD-L1, provide first success in some therapeutic settings; however, due to the complexity of the interactions between developing tumors and the immune system, the majority of cancer immunotherapies still need further improvement as proposed here by use of dynamic “Real-Time Tumor Vaccination”.

### TGF-Beta as a Key Driver of “Immune-Escape” in Advanced, Late-Stage Cancer

### Antagonization of the TGF-Beta System in Cancer

Human TGF-beta protein family consists preferably of three isotypes: TGF-beta1, TGF-beta2, and TGF-beta3 being differentially overexpressed in malignant tumors. Many recent studies favor the hypothesis that blocking TGF-beta-induced signaling in the tumor microenvironment enhances antitumor immunity and may be beneficial for cancer therapy (Jachimczak et al., 1993, Flavell et al., 2010). During the past two decades some important studies have highlighted the therapeutic potential of antagonizing the TGF-beta pathway in cancer (Akhurst et al., 2012). Many drugs that target TGF-beta function have been developed so far. Some of them have reached Phase III clinical trials for a number of disease indications including cancer (see Table 1). However, due to the complex biology of TGF-beta which is highly context-dependent and influenced by the cell type, disease stage in vivo and innate genetic variation among individuals, the development of the best strategy to target this system in humans is still very challenging (Connolly et al., 2012).

The major classes of TGF-beta inhibitors include ligand traps, antisense oligonucleotides (ASO), small molecule receptor kinase inhibitors and peptide aptamers (see e.g., Table 1). Ligand traps include anti-ligand neutralizing antibodies and soluble decoy receptor proteins that incorporate the ectodomains from either TGF-beta receptor II (TβRII) or TGF-beta receptor Ill (TβRIII)/betaglycan protein. Neutralizing antibodies have been raised against individual ligands or may be designed to block all three human isomers TGF-beta 1, -2 and -3. ASO can also be used to reduce the bioavailability of active TGF-beta ligands by blocking de novo synthesis of TGF-beta. ASOs are single-stranded polynucleotide molecules, usually 13-25 nucleotides in length, that hybridize to complementary RNA, inhibiting mRNA function, and preventing protein synthesis through accelerated mRNA degradation by RNase H or steric blockade. Another therapeutic strategy is to block TGF-beta receptor I (TβRI) activity through the use of small molecule receptor kinase inhibitors that act via ATP-competitive inhibition of the catalytic domain of the serine-threonine kinase of the TβRI (ALK5-Receptor). Lastly, targeting intracellular TGF-beta signaling molecules, such as Smads, is possible with the use of peptide aptamers. Aptamers are small peptide, DNA or RNA molecules containing a target-binding and a scaffolding domain that stabilize and interfere with the function of the target.

All anti-TGF-beta strategies described above suffer from several limitations with respect to their delivery, specificity and/or toxicity. However, their most crucial and common limitation by far is the fact that TGF-beta signaling is involved in many different normal physiological functions, and that normal tissues are as equally compromised as tumors upon systemic effects of anti-TGF-beta therapies. Therefore, the systemic neutralization of this otherwise so important growth factor is associated with the risk of severe side-effects. To overcome this issue, we invented local (i.e. intratumoral) application of the TGF-beta inhibitor in order to achieve efficient concentrations of the anti-TGF-beta drug in the growing tumor tissue, while the resulting systemic concentrations are far too low to cause any dose-limiting or clinically relevant toxicological effect (Hau & Jachimczak et al., 2007, Bogdahn et al., 2012).

### Real-Time Approach to Generate a Tumor Vaccine In Vivo (“Online Vaccine”)

The key element of the present invention involves “de novo” generation of effective anti-tumor immune response in cancer-bearing patients, characterized by a high antigenic specificity due to a continued scanning of the patient's personal tumor-associated antigens (pTAAs) profile in a real-time (“online scan”) in a TGF-beta-free environment. Such dynamic vaccination approach or in other words four-dimensional (4D) vaccine may be used to treat particularly advanced, late-stage, cancer by use of repetitive spacio (intratumoral)-temporal (real-time) therapy comprising or consisting of a TGF-beta Inhibitor with a particular combination of immunostimulating cytokines such as IL-2 and/or GM-CSF. Due to a local, intratumoral drug application the present invention overcomes all disadvantages of former combinations or pharmaceutical compositions for use in treating cancer and thus, provides a key impact for improved immunotherapy of advanced late-stage cancer. Although the side-effects associated with TGF-beta Inhibition are limited to a local region in close proximity to the tumor, the highly specific, TAA-driven anti-tumor immune response is amplified and systemically distributed across the body by the circulating T-cells. Thus, the advantages of the present invention, which employs an effective real-time approach to screen/scan intratumoral, all individually-specific TAA in a TGF-beta-free milieu, are that 1) the invention overcomes the disadvantages of former combinations or pharmaceutical compositions used to treat cancer and 2) the invention has a key impact on improved immunotherapy of advanced malignant cancer.

### SUMMARY

The present invention relates to a unique, spacio-temporal application of a combination of an immune-stimulating cytokine, e.g. at least two of immune-stimulating cytokines such as IL-2 and/or GM-CSF with a TGF-beta inhibitor such as an ALK5 inhibitor for use in a method of treating advanced, late-stage human cancer. In a first aspect the method involves INTRATUMORAL reduction of TGF-beta activity by blockade of TGF-beta signaling for example through the type I TGF-beta receptor kinase ALK5 (e.g., SD 208), wherein a local inhibition of a TGF-beta function in a growing tumor guides the immune system to scan all currently expressed personalized pTAAs in real-time by antigen-presenting cells (APC) from intratumoral lymphoid structures. In a second aspect, tumor tissue being exposed to sufficient TGF-beta inhibition activates intratumorally tertiary lymphoid structures to generate effective anti-tumor immune response. This includes both: induction of APC function by e.g. GM-CSF-treated CD103+ Dendritic Cells (DCs) as well as cytotoxic CD8+ T Lymphocytes by Interleukin 2-treated T cells in a TGF-beta-free melieu.

The simultaneous INTRATUMORAL application of both therapeutic regimens including TGF-beta Inhibition with immunostimulation e.g., by combined use of IL-2 and GM-CSF polarized the development of TGF-driven suppressive and/or toloregenic immune phenotype consisting of immunosuppressive T regulatory cells (Treg) and monocyte-derived suppressor cells (MDSC) results into a specific anti-tumor cytotoxic immune response.

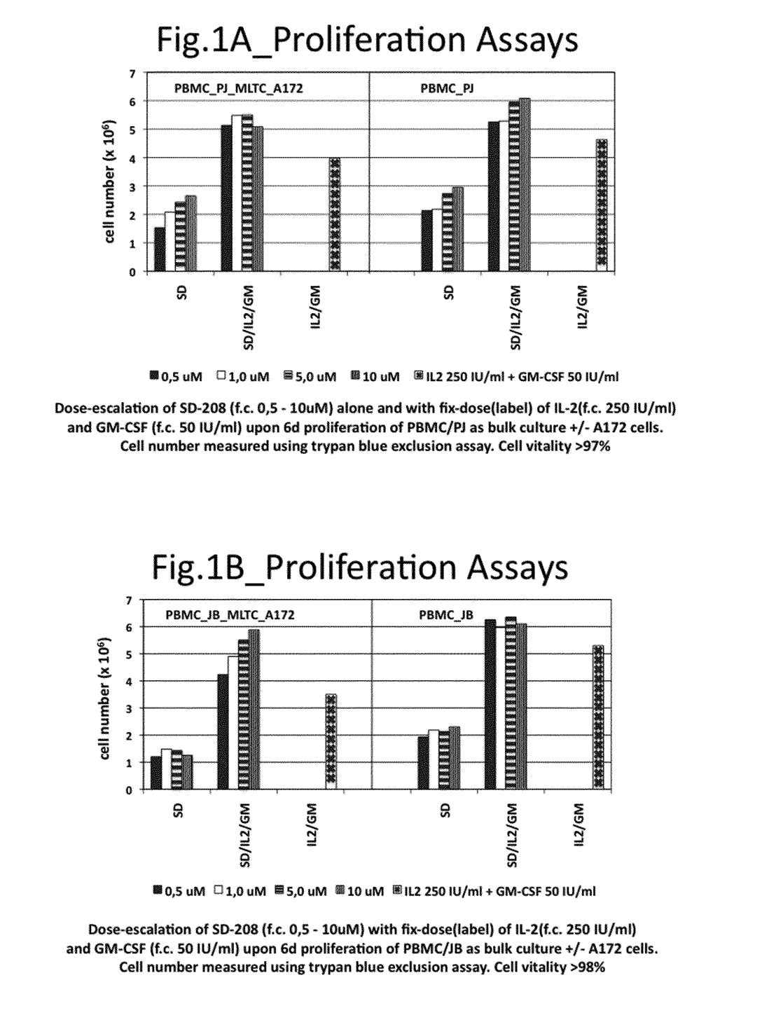

FIGS. 1A to 1C present the evaluation of growth-modulatory effects of SD208, IL-2 and GM-CSF upon PBMC proliferation, as measured with trypan blue exclusion assay.

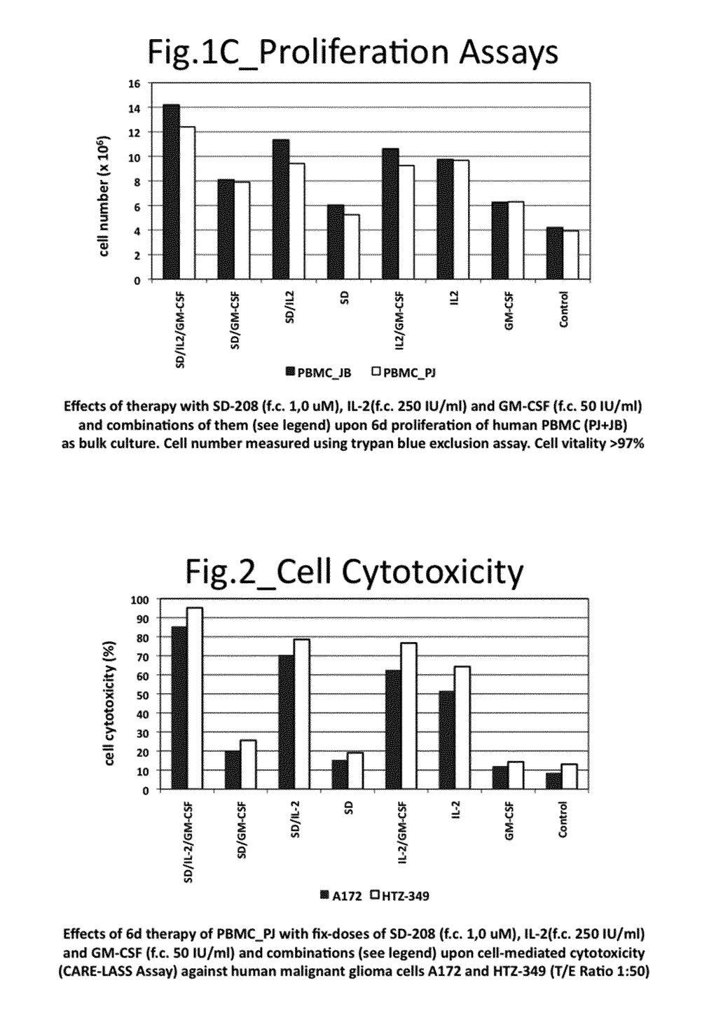

FIG. 2 shows the evaluation of effects of components of the present invention SD-208+IL-2+GM-CSF upon allogenous cell cytotoxicity against human glioma cells HTZ-349 and A-172 in a calcein-release-cell-cytotoxicity assay (CARE-LASS).

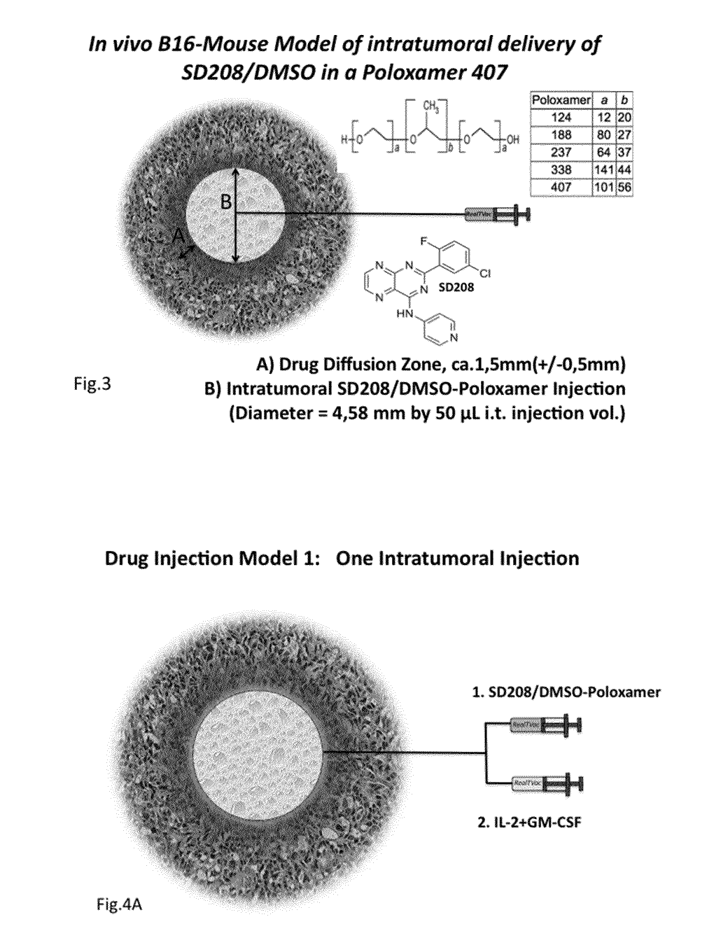

FIG. 3 presents an in vivo B16-mouse model of intratumoral delivery of SD208 in a poloxamer complex with DMSO.

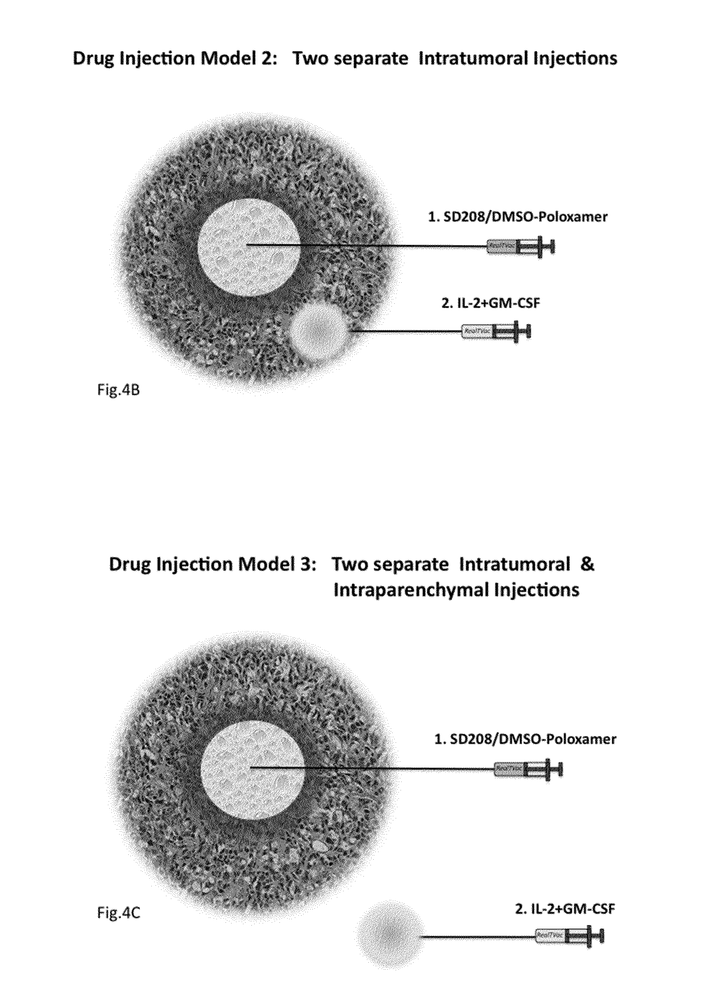

FIGS. 4A to 4C depict different models of intratumoral injection, wherein FIG. 4A refers to one intratumoral injection, FIG. 4B to two separate intratumoral injections and FIG. 4C to two separate intratumoral/intraparenchymal injections.

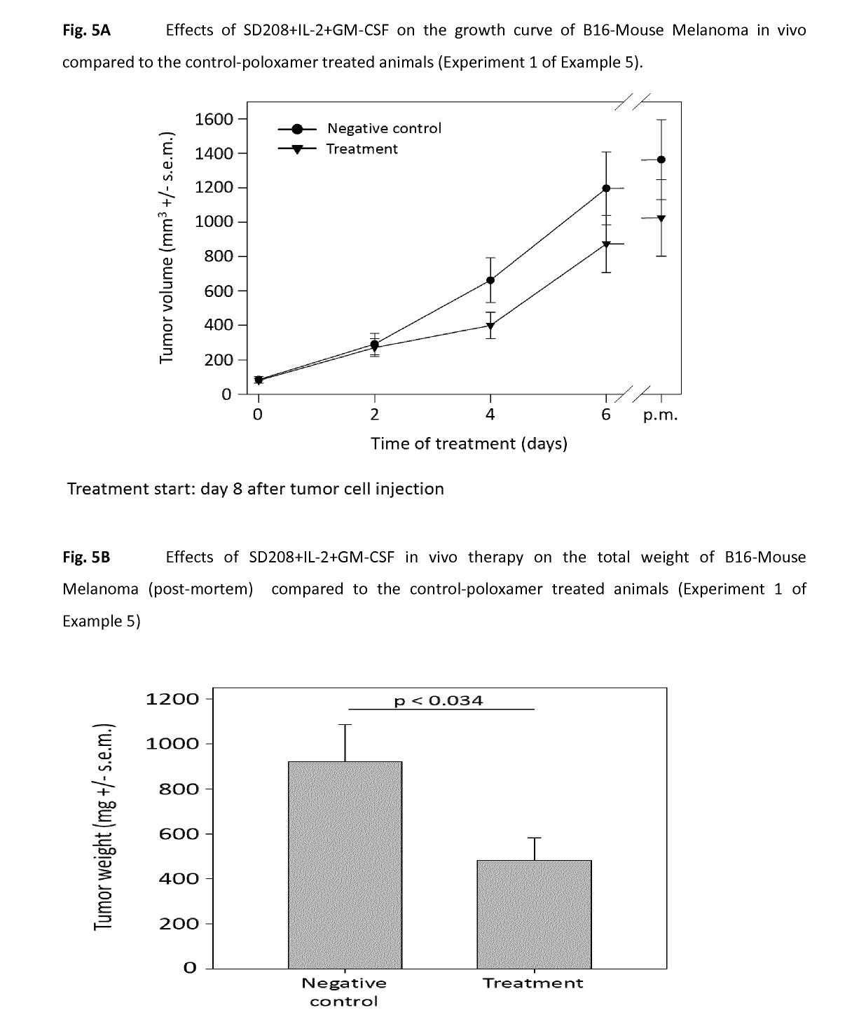

FIGS. 5A and 5B present an in vivo B16-mouse model treated with pharmaceutical components of RealTVac®, i.e., a combination of an ALK5 inhibitor such as SD208, IL-2 and GM-CSF. FIG. 5A presents the effects of the combination of SD208, IL-2 and GM-CSF on the tumor volume in the B16-mouse melanoma in comparison to a negative control treated with poloxamer which results in a reduction of the tumor volume of about 35%. FIG. 5B shows the effect of a combination of SD208, IL-2 and GM-CS on the total weight of B16-mouse melanoma compared to poloxamer treated control animals which results in about 45% reduction of the tumor weight.

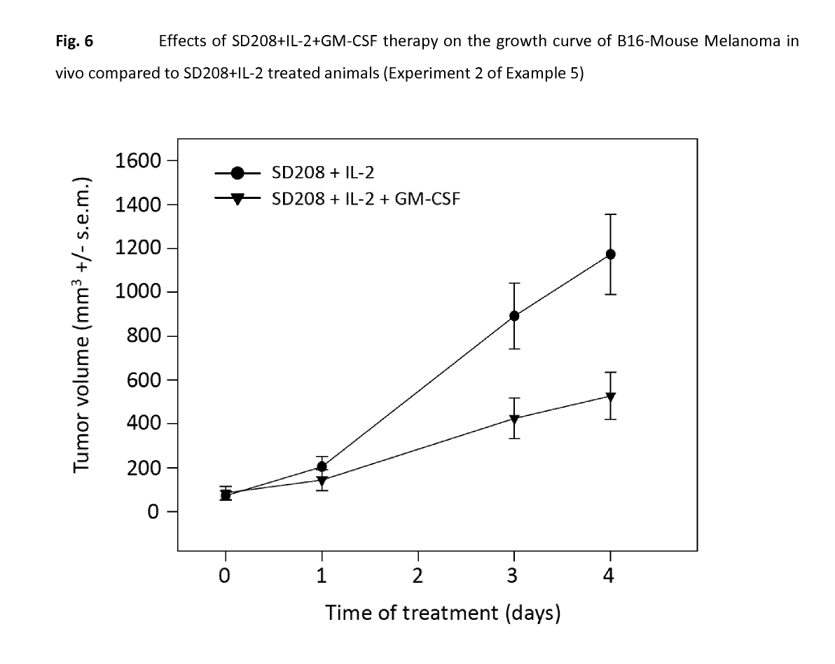

FIG. 6 shows the effects of a combination of SD208 and IL-2 in comparison to the effects of a combination of SD208, IL-2 and GM-CSF, wherein the combination of three compounds presents a stronger effect (about 50%).

### DETAILED DESCRIPTION

Essential elements of the present invention, which is a four-dimensional (4-D) approach for effective presentation of personal tumor associated antigens (pTAA) in vivo, is a combined administration in a spacial (intratumoral) and temporal (real-time) context of a TGF-beta inhibitor and one or more immune-stimulating cytokines, e.g. at least two. The four-dimensional (4-D) approach is defined as unification of space (three-dimensional tumor) and time (presentation of currently expressed pTAA profile in real-time mode, wherein the profile represents the phenotype of the tumor).

### 1) Inhibitor of TGF-Beta:

A TGF-beta inhibitor of the present invention interacts with a TGF-beta receptor, inhibits the signaling of the receptor, interacts with a TGF-beta protein, and/or inhibits the transcription and/or translation of the gene encoding TGF-beta-1, -2, and/or -3. The TGF-beta inhibitor of the present invention inhibits TGF-beta1, TGF-beta2, and/or TGF-beta3; in case an inhibitor inhibits all three TGF-beta isotypes, the inhibitor is a pan-specific inhibitor. Examples of TGF-beta inhibitors of the present invention are:

A) a small molecule inhibitor of TGF-beta receptor type I kinase (ALK5; an inhibitor is an ALK5 inhibitor),
B) a neutralizing anti-TGF-beta-1, -2, -3 antibody or TGF-beta binding fragments thereof,
C) a neutralizing anti-TGF-beta receptor type I, type II or type Ill antibody or TGF-beta receptors binding fragments thereof,
D) an antisense oligonucleotide specific for mRNA encoding TGF-beta1, -2, and -3 isotypes or other components of TGF-beta signaling assembly optionally comprising a modified nucleoside such as 2′-O, 4′-C-methylene linked bicyclic ribonucleotides, known as locked nucleic acids LNA (e.g., oxy-LNA, amino-LNA, thio-LNA), phosphorodiamidate morpholino oligomers (PMO), phosphorothioate (PS), 2′-O-methyl (2′-Ome), 2′-fluoro (2′-fluoro (2′-F), or 2′-methoxyethyl (2′-MOE) derivatives,
E) an antisense RNA molecule specific for TGF-beta2-mRNA like belagenpumatucel-L and/or TGF-beta1-mRNA or TGF-beta3-mRNA or other components of mRNA encoding TGF-beta signaling assembly,
F) a silencing RNA molecule (siRNA) specific for mRNA encoding TGF-beta1, -2, and/or -3 isotypes or other components of TGF-beta signaling assembly,
G) a short hairpin RNA (shRNA) specific for mRNA encoding TGF-beta1, -2, and/or -3 isotypes or other components of TGF-beta signaling assembly,
H) a miRNA molecule specific for mRNA encoding TGF-beta1, -2, and/or -3 isotypes or other components of TGF-beta signaling assembly,
I) an aptamer and/or spiegelmer molecule specific for TGF-beta1, -2, and/or -3 isotypes or other components of the TGF-beta signaling assembly, and/or
J) a ribozyme molecule specific for mRNA encoding TGF-beta1, -2, and/or -3 isotypes or other components of the TGF-beta signaling assembly.

ASOs are single-stranded polynucleotide molecules comprising 13-25 nucleotides, preferably 15-20 nucleotides, more preferred 15, 16, 17, 18, 19, 20, 21, 22 or 23 nucleotides, that hybridize to complementary RNA, inhibiting mRNA function, and preventing protein synthesis for example through accelerated mRNA degradation by RNase H or steric blockade.

In detail, a TGF-beta inhibitor binding to TGF-beta receptor type I, preferably a non-peptide, small molecule inhibitor of ALK-5 kinase from dihydropyrolopyrazole derivatives (e.g., LY2157299, LY21109761, LY580276, LY550410, GW788388), pyrazole derivatives (e.g., LY364947, HTS-466284, SM305, SB525334, A-83-01), quinazoline derivatives (e.g., SD-208), or imidazole derivatives (e.g., SM16, SB431542, or SB505124). SD-208 for example is a small molecule inhibitor of type I kinase of TGF-beta receptor with IC₅₀=49 nM, which inhibits TGF-beta signal transduction in a dose-dependent fashion, as for example measured by p-SMAD2 western blot analysis.

A human pan-anti-TGF-beta antibody binding to and neutralizing all three isotypes of TGF-beta (e.g., TGF-beta1, 2, 3) like GC-1008, a neutralizing high-affinity antibody or antigen-binding immunoglobulin single variable domain or polypeptide thereof, that neutralize mature human TGF-beta1 (like CAT 192), or hTGF-beta2 (like CAT 152), and neutralizing anti-TGF-beta receptor type II antibody (like D10) or an antibody fragment thereof which binds and neutralizes human TGF-beta receptor type II, a fusion protein comprising a TGF-beta type II receptor fusion protein, a small molecule or peptide consisting of, for example, 11-50 amino acid residues blocking assembly of the TGF-beta signaling complex extra- and intracellularly.

An amino acid-blocking assembly is a small polypeptide physically interacting with target protein(s) by electrostatic forces, leading to its (their) functional failure.

The present invention comprises administration of an effective amount of an iRNA agent, including sense and antisense sequences capable of forming an RNA duplex. The sense sequence of the iRNA agent preferably includes a nucleotide sequence substantially identical to a target sequence of about 19 to 23 nucleotides of TGF-beta-mRNA, and the antisense sequence preferably includes a nucleotide sequence complementary to a target sequence of about 19 to 23 nucleotides of TGF-beta mRNA. Further TGF-beta inhibitory drugs are summarized in Table 1:

TABLE 1

| Drug/Company | Type/Targets | Stage | Clinical indication |
|---|---|---|---|
| Trabedersen, Antisense Pharma | PS-ODNs TGF-β2 | Phase III | Malignant Cancer |
| Belagenpumatucel-L, NovaRx | AS-mRNA TGF-β2 | Phase III | NSCLC |
| Disitertide(P144), Digna Biotech | Peptide (TGFRIII fragment) | Phase II | Systemic Sclerosis |
| Lerdelimumab(CAT-152), Genzyme | Antibody TGF-β2 | Phase III | Glaucoma |
| Metelimumab (CAT-192), Genzyme | Antibidy TGF-β1 | Phase II | Systemic Sclerosis |
| Fresolumimab(GC-1008), Sanofi | Antibody TGF-β1/2/3 DNA | Phase I | Sclerosis, Cancer |
| FANG ™ Vaccine with bi-siRNAfurin | DNA Plasmid | Phase III | Malignant Cancer |
| LY2382770, Eli Lilly | Antibody TGF-β1 | Phase II | Kidney Fibrosis |
| SIX-1ββ, Stromedix | Antibody aVβ6 intergrin | Phase II | Fibrosis |
| Ly2157299, Eli Lilly | small molecule ALK5 TβRII | Phase II | Malignant Cancer |
| Dominant neg. TGFβR2-mod.T cells | Recombinant T cells | Phase I | Lung Cancer, Lymphoma, |
| Avotermin(Juvista), Renova | Recomb. Protein TGFβ3 | Phase II | Scarring |
| Pirfenidone, InterMune | Small Molecule TGF-β | Phase III | Fibrosis, Scarring |
| Losartan(AT1), Merck | Small Molecule TGF-β | Phase I/II | Marfan Syndrom |
| Tranilast, Kissei Pharmaceuticals | Small Molecule TGF-β | Phase III | Corneal Pterygium |
| IMC-TR1, ImClone/Eli Lilly | Antibody TGF-βRTII | Phase II | Malignant Cancer |
| AP11014, Antisense Pharma | PS-ODNs TGF-1 | Preclinical | Malignant Cancer |
| P17, Digna Biotech | Pept. binding TGF-βR/TGF | Preclinical | Fibrosis, Cancer |
| LSKL, Academic | Peptide Thrombospondin | Preclinical | Diabetic Nephropathy |
| 1D11, R&D Systems | Mouse pan-TGF-β-Antibody | Preclinical | Breast Cancer |
| SR2F, Academic | Ligand Trap TGF-β1 + β3 | Preclinical | Breast Cancer |
| soluble TβR2-Fc, Genzyme | Ligand Trap TGF-β1 + β3 | Preclinical | Breast Cancer |
| LY580276, LY550410, Eli Lilly | Small Molecule ALK5 TβRI | Preclinical | Cancer |
| SD-505124, SB-431542, GSK | Small Molecule ALK5 TβRI | Preclinical | Cancer |
| SD208, SD093, Scios | Small Molecule ALK5 TβRI | Preclinical | Cancer |
| Ki26894, Lirin Pharmaceuticals | Small Molecule ALK5 TβRI | Preclinical | Breast Cancer |
| SM16, Biogen Idec | Small Molecule ALK5 TβRI | Preclinical | Cancer |
| GW788388, GlaxoSmithKline | Small Molecule ALK5 TβRI | Preclinical | Fibrosis |
| GB1201, GB1203, Academic | Pyrrole-imidazole TGFβI | Preclinical | Fibrosis |
| ASPH_1047/0047, Isarna | LNA-ODNs TGF-β1/TGF-β2 | Preclinical | Cancer, Fibrosis |
| ALK5-Inhibitor, Rigel/BMS | Small molecule ALK5 TβRI | Preclinical | Cancer |

Table 1: Summary of TGF-beta inhibitory drugs in clinical and preclinical development according to Akhurst & Hata, 2012.

2) Immune-Stimulating Cytokines: Interleukin-2 (11-2) and/or Granulocyte-Macrophage Colony Stimulating Factor (GM-CSF)

IL-2 (Interleukin-2, aldesleukin) is a T helper1 (Th1) cytokine that plays a key role in the activation and proliferation of lymphocytes that have been primed by antigens (e.g, TAA). IL-2 is pivotal for the expansion of most T cells, NK cells, and B cells during various phases of their antigen response. IL-2 is secreted during T cell activation and fuels the growth of activated T cells. Apart from its most important role to mediate antigen-specific T lymphocyte proliferation, IL-2 also modulates the expression of IFNγ and MHC antigens, stimulates proliferation and differentiation of activated B cells, and augments NK cell activity. IL-2 signals through various signaling pathways that include the activation of JAK/STATS, RAS/MAPK and the PI3 kinase pathways. These activations make IL-2 a known potent T cell growth factor, inducer of lymphokine-activated killer activity, boosts the cytolytic activity of NK cells, augmentation of immune globulin (Ig)gt production, and an essential factor for the development of Treg cells (e.g., when used with TGB-beta).

GM-CSF (granulocyte-macrophage colony stimulating factor, such as sargramostin/molgramostin)(Leukine/USA-Leucomax/EU), is a pleiotropic, glycosylated growth factor produced by a wide variety of tissue types, including fibroblasts, endothelial cells, T cells, macrophages, mesothelial cells epithelial cells and various tumor types. The biological effects of GM-CSF are mediated through its binding to cell surface receptors that are widely expressed on hematopoietic cells, as well as some non-hematopoietic cells such as endothelial cells. GM-CSF stimulates proliferation, activation, and differentiation of myeloid dendritic cells (mDCs), intratumoral CD103+ dendritic cells, macrophages, granulocytes, neutrophils and eosinophils. GM-CSF can also enhance several T-cell stimulatory activities of DCs, including antigen uptake, major histocompatibility complex (MHC) activity, costimulatory molecule expression and interleukin 12 (IL-12) production. It may also enhance tumor invasion by upregulation of MMP-2 and -9 and -26 in cancer. Moreover, in TGF-beta environment, GM-CSF drives generation of MDSC. In most tissues, inflammatory mediators, such as IL-1, IL-6, TNFα, or endotoxin, are inducers of GM-CSF gene expression. GM-CSF was shown to be the most effective molecule in inducing antitumor reactivity (Dranoff 2003; Broz et al., 2014).

In a further embodiment, the present invention refers for example to a combination of IL-2 and/or GM-CSF with an ALK-5 inhibitor such as SD-208 for use in a method for the treatment of cancer, particularly in the local, e.g., intratumoral therapy of advanced, grade 3 or 4, late-stage and/or metastatic cancers such as melanoma, gastric carcinoma, colon carcinoma, rectal carcinoma, hepatocellular carcinoma, cholangiocarcinoma, pancreatic carcinoma, esophageal carcinoma, other adenocarcinoma, breast cancer, prostatic cancer, kidney cancer, non-small cell lung cancer, small cell lung cancer, retinoblastoma, ovarian carcinoma, cervix carcinoma, osteosarcoma, chondrosarcoma, leiomyosarcoma, rhabdomyosarcoma, mesothelioma, fibrosarcoma, angiosarcoma, liposarcoma, glioma, myxosarcoma, mixed connective tissue tumor, non-Hodgkin or Hodgkin lymphoma. Additionally, all combinations of the present invention are also for use in a method of treating metastasis of the above-mentioned malignant (primary) tumors.

The immune-stimulating cytokine such as IL-2 and/or GM-CSF and the ALK-5 inhibitor such as SD-208 are administered separately or combined in one product, for example, in a pharmaceutical composition, optionally comprising a pharmaceutically acceptable carrier. If the compounds are administered separately, they are administered consecutively, preferably in a defined time period, which can be e.g, 1 min. or 5 min. to 12 h, 30 min. to 6 h, or 1 to 2 h. In another alternative, the compounds of the combination, e.g., IL-2 and/or GM-CSF, and the ALK-5 inhibitor such as SD-208, are combined right before the intratumoral administration, and are administered for example in one injection. In an embodiment, all compounds of the combination or pharmaceutical composition show their treating effects at the same time.

The intratumoral concentration of the immune stimulator IL-2 is for example in a range of 1.8×10⁶ to 18×10⁶ IU, and/or the intratumoral concentration of the immune stimulator GM-CSF is in a range of 0.1-0.5 mg; in such combination, the intratumoral concentration of the ALK5 inhibitor is for example in a range of about 1 nM to about 10 mM, or 0.1 μM to 1 mM.

The immune stimulator, e.g., IL-2, GM-CSF, and the TGF-beta inhibitor, e.g. ALK-5 inhibitor are optionally combined with another TGF-beta-specific DNA- and RNA-targeting compound such as siRNA, shRNA, ribozymes, miRNA, antisense RNA, aptamer, antisense oligonucleotides, spiegelmer etc. All these compounds are administered subsequently, wherein one or more of the compound(s) is administered every day, every week, every month, every three months, every six months, and one or more other compounds are administered every day, every week, every month, every three months, every six months, i.e., in a time lag to the administration of the first, second or third etc. compound. Alternatively, all compounds are administered at the same time, e.g., in the form of a pharmaceutical composition comprising or consisting of the compounds, and optionally comprising a pharmaceutically acceptable carrier. Just like for each compound, the pharmaceutical composition is preferably administered intratumorally.

In another embodiment of the present invention, an additional immunomodulating factor is combined with IL-2 and/or GMCSF and a TGF-beta inhibitor. The additional immunomodulating factor is selected from the following group consisting of:

A) a further immune-stimulating cytokine: IFNα, IFNβ, IFNγ, MIPα, MIPβ, TNFα, G-CSF, M-CSF, MIP, and interleukins (IL) like IL-1α, IL-1β, IL-3, IL-5, IL-6, IL-7, IL-12, IL-15, IL-16, IL-18, IL-21, IL-24, IL-32, IL-33, IL-36 which likewise regulate the function of the immune system,
B) a tumor associated antigen (TAA) such as unique antigens resulting from mutations as presented for example in Table 2, tumor-specific antigens such as presented for example in Table 3, differentiation antigens such as presented for example in Table 4, or overexpressed antigens such as those presented for example in Table 5.
C) an immune-stimulating ligand binding to and activating costimulatory receptors expressed on immune cells, for example, CD40, CD134 (OX40), CD137, or TIM-3,
D) an inhibitor of prostaglandin E (PGE), interleukin 10 (IL-10), and interleukin 35 (IL-35),
E) an inhibitor of immunoglobulin-like transcript (ILT) such as e.g, ILT2 and ILT3,
F) an inhibitor of immunomodulating cytotoxic T-lymphocyte antigen 4 (CTLA-4), e. g. ipilimumab,
G) an inhibitor of glucocorticoid-induced tumor necrosis factor family receptor (GITR),
H) an inhibitor of programmed cell death protein 1 (PD-1), such as e.g, nivolumab or pembrolizumab and respective ligands, i.e. other molecules binding the protein e.g. PD-L1 and/or PD-L2, and/or
I) an inhibitor of melanoma inhibiting activity (MIA).

TABLE 2

| | Tumor Antigens Resulting from Mutations |
|---|---|
| date of download: 20140309 | http://cancerimmunity.org/peptide/mutations/ |
| Gene/Protein | Peptide |
| alpha-actinin-4 | FIASNGVKLV |
| ARTC1 | YSVYFNLPADTIYTNh |
| BCR-ABL fusion protein (b3a2) | SSKALQRPV, GFKQSSKAL, ATGFKQSSKALQRPVAS |
| B-RAE | EDLTVKIGDFGLATEKSRWSGSHQFEQLS |
| CASP-5 | FLIIWQNTM |
| CASP-8 | FPSDSWCYF |
| beta-catenin | SYLDSGIHF |
| Cdc27 | FSWAMDLDPKGA |
| CDK4 | ACDPHSGHFV |
| CDKN2A | AVCPWTWLR |
| CLPP | ILDKVLVHL |
| COA-1 | TLYQDDTLTLQAAG |
| dek-can fusion protein | TMKQICKKEIRRLHQY |
| EFTUD2 | KILDAVVAQK |
| Elongation factor 2 | ETVSEQSNV |
| ETV6-AML1 fusion protein | RIAECILGMi, IGRIAECILGMNPSR |
| FLT3-ITD | YVDFREYEYY |
| FN1 | MIFEKHGFRRTTPP |
| GPNMB | TLDWLLQTPK |
| LDLR-fucosyltransferaseAS fusion protein | WRRAPAPGA, PVTWRRAPA |
| hsp70-2 | SLFEGIDIYT, AEPINIQTW |
| MART2 | FLEGNEVGKTY |
| ME1 | FLDEFMEGV |
| MUM-1f | EEKLIVVLF |
| MUM-2 | SELFRSGLDSY |
| MUM-3 | EAFIQPITR |
| neo-PAP | RVIKNSIRLTL |
| Myosin class I | KINKNPKYK |
| NFYC | QQITKTEV |
| OGT | SLYKFSPFPL |
| OS-9 | KELEGILLL |
| p53 | VVPCEPPEV |
| pml-RARalpha fusion protein | NSNHVASGAGEAAIETQSSSSEEIV |
| PRDX5 | LLLDDLLVSI |
| PTPRK | PYYFAAELPPRNLPEP |
| K-ras | VVVGAVGVG |
| N-ras | ILDTAGREEY |
| RBAF600 | RPHVPESAF |
| SIRT2 | KIFSEVTLK |
| SNRPD1 | SHETVIIEL |
| SYT-SSX1 or -SSX2 fusion protein | QRPYGYDQIM |
| TGF-betaRII | RLSSCVPVA |
| Triosephosphate isomerase | GELIGILNAAKVPAD |

TABLE 3

| | Shared Tumor-Specific Antigens |
|---|---|
| date of download: 20140309 | http://cancerimmunity.org/peptide/tumor-specific/ |
| Gene | Peptide |
| BAGE-1 | AARAVFLAL |
| Cyclin-A1 | SLIAAAAFCLA, FLDRFLSCM |
| GAGE-1,2,8 | YRPRPRRY |
| GAGE-3,4,5,6,7 | YYWPRPRRY |
| GnTVf | VLPDVFIRC(V) |
| HERV-K-MEL | MLAVISCAV |
| KK-LC-1 | RQKRILVNL, NYNNFYRFL |
| KM-HN-1 | EYLSLSDKI, EYSKECLKEF, MLMAQEALAFL, |
| LAGE-1 | SLLMWITQC, MLMAQEALAFL, LAAQERRVPR, LELVRRILSR, |
| | APRGVRMAV, QGAMLAAQERRVPRAAEVPR, ILSRDAAPLPRPG |
| | AADHRQLQLSISSCLQQL, SLLMWITQCFLPVF, |
| | CLSRRPWKRSWSAGSCPGMPHL, AGATGGRGPRGAGA |
| | AGATGGRGPRGAGA |
| MAGE-A1 | EADPTGHSY, KVLEYVIKV, SLFRAVITK, EVYDGREHSA, |
| | RVRFFFPSL, EADPTGHSY, REPVTKAEML, KEADPTGHSY, |
| | SAFPTTINF, DPARYEFLW, ITKKVADLVGF, SAYGEPRKL |
| | RVRFFFPSL, SAYGEPRKL, TSCILESLFRAVITK, EYLQLVFGI |
| | TSCILESLFRAVITK, PRALAETSYVKVLEY, YLQLVFGIEV, |
| | FLLLKYRAREPVTKAE, EYVIKVSARVRF, EYVIKVSARVRF, |
| MAGE-A2 | REPVTKAEML, EGDCAPEEK, EYLQLVFGI,LLKYRAREPVTKAE, |
| | EVDPIGHLY, FLWGPRALV, KVAELVHFL, TFPDLESEF, |
| MAGE-A3 | VAELVHFLL, MEVDPIGHLY, EVDPIGHLY, MEVDPIGHLY |
| | REPVTKAEML, AELVHFLLL, WQYFFPVIF, EGDCAPEEK |
| MAGE-A3 | KKLLTQHFVQENYLEY, RKVAELVHFLLLKYR, VIFSKASSSLQL |
| | KKLLTQHFVQENYLEY, ACYEFLWGPRALVETS |
| | RKVAELVHFLLLKYR, VIFSKASSSLQL, FLLLKYRAREPVTKAE, |
| | VFGIELMEVDPIGHL, GDNQIMPKAGLLIIV |
| | TSYVKVLHHMVKISG, RKVAELVHFLLLKYRA |
| MAGE-A4 | EVDPASNTY, GVYDGREHTV, NYKRCFPVI, SESLKMIF, |
| MAGE-A6 | EVDPIGHVY, REPVTKAEML, EGDCAPEEK, ISGGPRISY |
| | LLKYRAREPVTKAE |
| MAGE-A9 | ALSVMGVYV |
| MAGE-A10 | GLYDGMEHLI, DPARYEFLW |
| MAGE-Al2 | EGDCAPEEK, REPFTKAEMLGSVIR, VRIGHLYIL, FLWGPRALV, |
| | AELVHFLLLKYRAR, |
| MAGE-C1 | SSALLSIFQSSPE, ILFGISLREV, SFSYTLLSL, VSSFFSYTL, |
| | KVVEFLAML, |
| MAGE-C2 | LLFGLALIEV, SSTLYLVFSPSSFST, ASSTLYLVF, SESIKKKVL |
| | ALKDVEERV, |
| mucin | PDTRPAPGSTAPPAHGVTSA |
| NA88-A | QGQHFLQKV |
| NY-ESO-1/LAGE-2 | LAAQERRVPR, TVSGNILTIR, APRGPHGGAASGL |
| | MPFATPMEA, KEFTVSGNILTI, LAMPFATPM, MLMAQEALAFL, |
| | ARGPESRLL, SLLMWITQCFLPVF, SLLMWITQC, ASGPGGGAPR |
| | LLEFYLAMPFATPMEAELARRSLAQ, RLLEFYLAMPFA |
| | PGVLLKEFTVSGNILTIRLTAADHR, PFATPMEAELARR |
| | QGAMLAAQERRVPRAAEVPR, PGVLLKEFTVSGNILTIRLT |
| | VLLKEFTVSG, AADHRQLQLSISSCLQQL, KEFTVSGNILT |
| | LKEFTVSGNILTIRL, LLEFYLAMPFATPM, AGATGGRGPRGAGA |
| SAGE | LYATVIHDI |
| Sp17 | ILDSSEEDK |
| SSX-2 | KASEKIFYV, EKIQKAFDDIAKYFSK, FGRLQGISPKI |
| | WEKMKASEKIFYVYMKRK, KIFYVYMKRKYEAMT |
| SSX-4 | INKTSGPKRGKHAWTHRLRE, KHAWTHRLRERKQLVVYEEI |
| | YFSKKEWEKMKSSEKIVYVY, KSSEKIVYVYMKLNYEVMTK |
| | MKLNYEVMTKLGFKVTLPPF, LGFKVTLPPFMRSKRAADFH |
| | KHAWTHRLRERKQLVVYEEI |
| TAG-1 | SLGWLFLLL, LSRLSNRLL |
| TAG-2 | LSRLSNRLL |
| TRAG-3 | CEFHACWPAFTVLGE |
| TRP2-INT2g | EVISCKLIKR, RQKKIRIQL |
| XAGE-1b/GAGED2a | HLGSRQKKIRIQLRSQ, CATWKVICKSCISQTPG |

TABLE 4

| | Differentiation Antigens |
|---|---|
| date of download: 20140309 | http://cancerimmunity.org/peptide/differentiation/ |
| Gene/Protein | Peptide |
| CEA (gut carcinoma) | YLSGANLNL, IMIGVLVGV, GVLVGVALI, HLFGYSWYK, |
| | DTGFYTLHVIKSDLVNEEATGQFRV, YSWRINGIPQQHTQV, |
| | QYSWFVNGTF, TYYRPGVNLSLSC, EIIYPNASLLIQN, |
| | TYACFVSNL,YACFVSNLATGRNNS, LWWVNNQSLPVSP, |
| | AYVCGIQNSVSANRS, EIIYPNASLLIQN, NSIVKSITVSASG |
| gp100/Pme117 (melanoma) | KTWGQYWQV, (A)MLGTHTMEV, ITDQVPFSV, |
| | YLEPGPVTA, LLDGTATLRL, VLYRYGSFSV, SLADTNSLAV, |
| | RLMKQDFSV, RLPRIFCSC, LIYRRRLMK, ALLAVGATK |
| | IALNFPGSQK, ALNFPGSQK, VYFFLPDHL, RTKQLYPEW |
| | HTMEVTVYHR, SSPGCQPPA, VPLDCVLYRY, LPHSSSHWL, |
| | LPHSSSHWL, SNDGPTLI, GRAMLGTHTMEVTVY, |
| | WNRQLYPEWTEAQRLD, TTEWVETTARELPIPEPE, |
| | TGRAMLGTHTMEVTVYH, GRAMLGTHTMEVTVY |
| mammaglobin-A(breast cancer) | PLLENVISK |
| Melan-A / MART-1 | AEEAAGIGIL(T), RNGYRALMDKS, (E)AAGIGILTV, |
| (melanoma) | YTTAEEAAGIGILTVILGVLLLIGCWYCRR, EAAGIGILTV, |
| | ILTVILGVL, EEAAGIGILTVI, AAGIGILTVILGVL, |
| | APPAYEKLpSAEQ, MPREDAHFIYGYPKKGHGHS, |
| | RNGYRALMDKSLHVGTQCALTRR, EEAAGIGILTVI, |
| | KNCEPVVPNAPPAYEKLSAE |
| NY-BR-1 (breast cancer) | SLSKILDTV |
| OA1 (melanoma) | LYSACFWWL |
| PAP (prostate cancer) | FLFLLFFWL, TLMSAMTNL, ALDVYNGLL |
| PSA (prostate carcinoma) | FLTPKKLQCV, VISNDVCAQV |
| RAB38/NY-MEL-1 (melanoma) | VLHWDPETV |
| TRP-1/gp75 ( melanoma) | MSLQRQFLR, SQWRVVCDSLEDYDT, SLPYWNFATG, |
| | ISPNSVFSQWRVVCDSLEDYD |
| TRP-2 (melanoma) | SVYDFFVWL, TLDSQVMSL, LLGPGRPYR, ANDPIFVVL, |
| | QCTEVRADTRPWSGP, ALPYWNFATG |
| tyrosinase (melanoma) | KCDICTDEY, SSDYVIPIGTY, MLLAVLYCL, CLLWSFQTSA, |
| | YMDGTMSQV, AFLPWHRLF, IYMDGTADFSF, |
| | TPRLPSSADVEF, LPSSADVEF, LHHAFVDSIF, |
| | QNILLSNAPLGPQFP, SYLQDSDPDSFQD, QCSGNFMGF |
| | FLLHHAFVDSIFEQWLQRHRP, SEIWRDIDF |

TABLE 5

| | Antigens Overexpressed in Tumors |
|---|---|
| date of download: 20140309 | http://cancerimmunity.org/peptide/oyerexpressed/ |
| Gene | Peptide |
| VLRENTSPK | SVASTITGV |
| AIM-2 | RSDSGQQARY |
| ALDH1A1 | LLYKLADLI |
| BCLX (L) | YLNDHLEPWI |
| BING-4 | CQWGRLWQL |
| CALCA | VLLQAGSLHA |
| CD45 | KFLDALISL |
| CPSF | KVHPVIWSL, LMLQNALTTM |
| cyclin D1 | LLGATCM FV, NPPSMVAAGSVVAAV |
| DKK1 | ALGGHPLLGV |
| ENAH (hMena) | TMNGSKSPV |
| EpCAM | RYQLDPKFI |
| EphA3 | DVTFNIICKKCG |
| EZH2 | FMVEDETVL, FINDEIFVEL, KYDCFLHPF, KYVGIEREM, |
| FGF5 | NTYASPRFK |
| glypican-3 | FVGEFFTDV, EYILSLEEL, |
| G250/MN/CAIX | HLSTAFARV |
| HER-2/neu | KIFGSLAFL, IISAVVGIL, ALCRWGLLL, ILHNGAYSL, |
| | RLLQETELV, VVLGVVFGI, YMIMVKCWMI, HLYQGCQVV, |
| | YLVPQQGFFC, PLQPEQLQV, TLE EITGYL, ALIHHNTHL, |
| | PLTSIISAV, VLRENTSPK, TYLPTNASL |
| IDO1 | ALLEIASCL |
| IGF2B3 | NLSSAEVVV, RLLVPTQFV |
| IL13Ralpha2 | WLPFGFILI |
| Intestinal carboxyl esterase | SPRWWPTCL |
| alpha-foetoprotein | GVALQTMKQ, FMNKFIYEI, QLAVSVILRV, |
| Kallikrein 4 | FLGYLILGV, SVSESDTIRSISIAS, LLANGRMPTVLQCVN, |
| | RMPTVLQCVNVSVVS |
| KIF20A | LLSDDDVVV, AQPDTAPLPV, CIAEQYHTV, |
| Lengsin | FLPEFGISSA |
| M-CSF | LPAVVGLSPGEQEY |
| MCSP | VGQDVSVLFRVTGALQ |
| mdm-2 | VLFYLGQY |
| Meloe | TLNDECWPA, FGRLQGISPKI, CPPWHPSERISSTL |
| MMP-2 | GLPPDVQRV |
| MMP-7 | SLFPNSPKWTSK |
| MUC1 | STAPPVHNV, LLLLTVLTV, PGSTAPPAHGVT |
| MUC5AC | TCQPTCRSL |
| p53 | LLGRNSFEV, SQKTYQGSY, PGTRVRAMAIYKQ, RMPEAAPPV |
| | HLIRVEGNLRVE |
| PAX5 | TLPGYPPHV |
| PBF | CTACRWKKACQR |
| PRAME | VLDGLDVLL, SLYSFPEPEA, ALYVDSLFFL, SLLQHLIGL, |
| PSMA | NYARTEDFF |
| RAGE-1 | LKLSGVVRL, PLPPARNGGL, SPSSNRIRNT |
| RGS5 | LAALPHSCL, GLASFKSFLK |
| RhoC | RAGLQVRKNK |
| RNF43 | ALWPWLLMA(T), NSQPVWLCL |
| RU2AS | LPRWPPPQL |
| secernin 1 | KMDAEHPEL |
| SOX10 | AWISKPPGV, SAWISKPPGV |
| STEAP1 | MIAVFLPIV, HQQYFYKIPILVINK |
| survivin | ELTLGEFLKL, TLGEFLKLDRERAKN, |
| Telomerase | ILAKFLHWL, RLVDDFLLV, LTDLQPYMRQFVAHL, |
| | RPGLLGASVLGLDDI |
| VEGF | SRFGGAVVR |
| WT1 | TSEKRPFMCAY, CMTWNQMNL, LSHLQMHSRKH, |
| | KRYFKLSHLQMHSRKH |

In a further embodiment, a pharmaceutical composition comprises poloxamers in addition such as poloxamer 124, 188, 237, 338, 388 and 407, which e.g., represent a pharmaceutically acceptable carrier. Poloxamers are nonionic triblock copolymers consisting of a central hydrophobic chain of polyoxypropylene (PPO) flanked by two hydrophilic chains of polyoxyethylene (PEO). Poloxamers favour the advantage of a concentration and temperature dependent micelle formation, thermoviscosification and/or gelation. Depending on the PEO and PPO content, selected Poloxamers form injectable sols at temperatures near or below ambient temperature, but exhibit gelation at the body temperature in concentrated solutions that provides the required retardation. In our experiments, a series of Poloxamers of different compositions were extensively investigated regarding their suitability for SD-208 retardation. The formulation technique was systematically optimized. Gel formation, gel stability depending on the environmental conditions, inclusion of drug and injectability were confirmed. Poloxamers are added to stabilize the immune-stimulating cytokine such as IL-2 and GM-CSF, and/or the TGF-beta inhibitor such as ALK-5, and thus, to extend their effect on their target. In another alternative, the combination with one or more poloxamers is for example the basis for a retard formulation, where from the TGF-beta inhibitor and/or the immune-stimulating cytokine and/or the immune-modulating compound are/is released during a certain time period, for example during 12 or 24 h.

### Intratumoral Treatment

In an embodiment, the immune-stimulating cytokine and/or the TGF-beta inhibitor or the pharmaceutical composition, comprising or consisting of these compounds of the present invention, are administered intratumorally. As described (see Background), the TAA phenotype of every individual tumor appears to be the result of genomic instability that generates a unique personal TAA profile. Thus, intratumoral application of drugs for activating the host immune system guides the patients's own tumor to serve as a source of currently expressed, unique TAA profile, i.e., cancer profile. Evaluation of the continuously changing profile of TAA, i.e., of a dynamic TAA profile in a rapidly growing tumor in vivo for the purpose of vaccine preparation is technically and logistically impossible. In the present invention, the tumor itself is used to present in real-time the full range of personal TAA in a TGF-beta-free context. Thus, the combined use, e.g., in a pharmacological composition, of a TGF-beta inhibitor in combination with immunostimulating cytokines IL-2 and/or GM-CSF triggers the induction of an antigen-specific immune response against currently available TAA. Intratumoral approach to use a pharmaceutical composition of the present invention generates not only a local anti-tumor immune response but also a systemic reaction by circulating T cells. For example employing immune-stimulating cytokine IL-2 and/or GM-CSF and a TGF-beta inhibitor such as SD-208, e.g., in a pharmaceutical composition, as described, generates not only a local immune response, but indeed also a systemic immune response to the TAA profile in the secondary lesion (metastasis) (Mahwi et al., 2007). Due to a continuous Darwinian selection process in malignant progression of cancer, intratumoral therapy with a pharmaceutical composition, as described in the present invention, is preferably applied repeatedly, whilst always accompanying—in real-time mode—all changes occurring in the TAA profile of the malignant tumor.

Another important aspect of the current invention is closely related to the significant toxicity associated with systemic application of TGF-beta inhibitors. The vast majority of current drug development programs strongly focus on systemic drug application, mainly driven by the intention to treat disease targets spread throughout the whole body. This also applies to the different systemic approaches, which are currently under investigation to treat cancers by use of anti-TGF-beta therapies, including inhibitors of TGF-beta receptors, TGF-beta targeting antibodies, ligand traps or Inhibitors of TGF-beta synthesis (see e.g., Table 1). All these strategies suffer from several limitations with respect to delivery, specificity and/or toxicity. However, their most crucial and common limitation by far is the fact that TGF-beta signaling is involved in many different normal physiological functions, and that normal tissues are as equally compromised as tumors upon systemic effects of anti-TGF-beta therapies. Therefore, the systemic neutralization of this otherwise so important protein is associated with the risk of severe side-effects. In addition, a systemic delivery of TGF-beta inhibitors may hinder the dosing parameters from being maximized because of the potentially harmful side effects. TGF-beta inhibitors may exert their activity in a variety of ways in the treatment of malignant cancer. In most instances, the TGF-beta inhibitor may not target the abnormal cell specifically, but rather exert its activity systemically across all cells. Systemic administration may expose, therefore, both transformed and healthy normal cells to the effects of the TGF-beta inhibitor. Although potentially therapeutically effective against the cancer cells in many ways, systemic administration of TGF-beta inhibitors may cause detrimental side effects to the normal healthy cells. This may result in a smaller amount of the intended dosage of TGF-beta inhibitors reaching and targeting the cancer cells. In addition, a greater amount of the intended dosage of TGF-beta inhibitors may reach the normal healthy cells. Thus, systemic delivery of TGF-beta inhibitors may hinder the dosing parameters from being maximized because of the potentially harmful side effects. The current invention of intratumoral application of a pharmaceutical formulation allows high concentrations of drugs to be achieved within the tumor, whereby this is essential to attain the desired therapeutic effects whilst avoiding systemic toxicity due to overdosing. Preferred concentrations of TGF-beta inhibitors, particularly of ALK-5 inhibitors are for example from about 1 nM to about 10 mM, from about 10 nM to about 10 mM, from about 0.1 μM to about 1 mM, or from about 500 nM to about 5 mM.

To overcome this issue, we invented local (i.e. intratumoral) application of the TGF-beta inhibitor such that efficient concentrations of the anti-TGF-beta drug can be achieved in the growing tumor, while the resulting systemic concentrations are far too low to cause any dose-limiting or clinically relevant toxicity (FIG. 4). The current invention of intratumoral application of the pharmaceutical composition allows achieving high concentrations of drugs within the tumor, which is essential to attain the desired therapeutic effects, while avoiding systemic toxicity due to overdosing. Nevertheless, the real-time vaccination approach can still result in a systemic anti-tumor response by a highly specific, TAA-driven immune response that is amplified and systemically distributed across the body by the circulating T-cells.

The major advantage of the present invention is the combination of simultaneous reduction in TGF-beta function with concomitant activation of the immune cells with e.g., IL-2 and/or GM-CSF. Thus, each individual immune system is able to adapt to the specific tumor situation at the time of the treatment, i.e., in real-time mode and is not limited to pre-selected tumor antigens, as applied in the classic tumor vaccine approach. This is particularly important, as malignant tumors rapidly change their antigenic profile due to a Darwinian selection process. In consequence, the present invention of a combination therapy comprising or consisting of an immunostimulating cytokine, e.g., at least two immune-stimulating cytokines, and a TGF-R inhibitor, when applied intratumorally, always induces maximal therapeutic effect due to the generation of an up-to-date host immune response to the growing tumor. In a one embodiment, at least two immune-stimulating cytokines such as IL-2 and GMCSF are combined with an ALK-5 inhibitor such as SD208. In another embodiment at least two immune-stimulating cytokines are combined with a TGF-beta inhibitor whereof one is IL-2 or GM-CSF.

In a further embodiment, the transcription and translation of mRNA, respectively, encoding TGF-beta as well as the interaction of TGF-beta ligands with its receptor strengthen the effect of the immune-stimulating cytokines such as IL-2 and GM-SCF. Further immune stimulators supporting the effect of IL-2 and/or GM-SCF are selected from the group consisting of neoantigens, other immune-stimulating cytokines such as IFNα, IFNβ, IFNγ, MIPα, MIPβ, TNFα, G-CSF, M-CSF, MIP, and interleukins (IL) such as IL-1α, IL-1β, IL-3, IL-5, IL-6, IL-7, IL-12, IL-15, IL-16, IL-18, IL-21, IL-24, IL-32, IL-33, IL-36, immune-regulating molecules binding to CD40, CD134 (OX40) or CD137, CTLA-4, PD-1, PD-L1 and PD-L2, and/or inhibitors of immune-inhibiting factors such as PGE, IL-10, IL-35, ILT2, ILT3 and/or GITR.

The reduction of the tumor volume and the tumor weight, respectively, by a combination of the present invention is for example in the range of 10 to 90%, 20 to 80%, 30 to 60% or 40 to 50%. In some embodiments the reduction is 35 or 45%.

For the purpose of clarity and a concise description, features are described herein as part of the same or separate embodiment. However, it should be understood that the scope of the invention may include embodiments having combinations of all or some of the features described.

### EXAMPLES

In the following, examples are provided to specify some aspects of the present invention, but these examples are not considered to limit the scope of the present invention, and the invention is not bound to these examples.

### Example 1

Establishment and culture of primary tumor cell cultures were performed as previously described (Jachimczak et al., 1993). Briefly, tumor specimens were cultivated in Dulbecco's minimum essential medium (DMEM) containing 10% FCS, 1% BME vitamin solution, and 1% MEM nonessential amino acids. HTZ-349 was established as primary cell culture from a patient suffering from glioblastoma multiforme (GBM, Grade IV). A-172 cells were obtained from ATCC (GBM). Peripheral blood mononuclear cells (PBMC) were isolated from venous blood of two healthy donors by standard Ficoll-Hypaque gradient centrifugation (Jachimczak et al., 1993).

SD-208, a small molecule inhibitor of type I kinase of TGF-beta receptor with IC₅₀=49 nM, was purchased from Sigma, (Germany). SD-208 inhibits TGF-beta signal transduction in a dose-dependent fashion, as measured by p-SMAD2 western blot analysis (Gaspar et al., 2007).

Interleukin 2 (IL-2, Proleukin S, Aldesleukin), used in this study is a human highly purified protein with a molecular weight of approximately 15,300 daltons, produced by recombinant DNA technology in a genetically engineered E. coli strain, purchased from Novartis (Germany). Proleukin S with a specific activity of 1.1 mg=18 million IU was reconstituted in sterile water to a final concentration of 10⁷ UI/1 ml for further use.

Granulocyte macrophage-colony stimulating factor (GM-CSF, CellGro) is a human recombinant, highly purified protein with a molecular weight of approximately 14.400 daltons produced by recombinant DNA technology using genetically engineered GM-CSF with a specific activity of 50 μg=4×10⁵ IU; this material was purchased from CellGenix GmbH (Germany). CellGro was reconstituted in sterile PBS to a final concentration of 50 μg/1 ml for further use.

### Example 2

The growth-modulatory effects of various concentrations (SD-208: 0.5-10 μM) were investigated employing a standard proliferation assay (Trypan Blue exclusion). FIGS. 1 A-C show the relative growth of PBMC treated for 6 days with IL-2, GM-CSF and different concentrations of SD-208. The growth stimulatory effects observed by combined application of IL-2 and GM-CSF were further enhanced by simultaneous treatment with the TGF-beta inhibitor, SD-208.

### Example 3

Cell-mediated tumor cytotoxicity assay (CARE-LASS): In order to evaluate the cytotoxic potential of immune cells treated with TGF-beta inhibitor and immunostimulating cytokines IL-2 and GM-CSF, both effector PBMC and glioma target cells were either preincubated with SD-208 and cytokines or left untreated for 6 days. PBMC were activated with IL-2 (Proleukin S) and GM-CSF. Immune cell cytotoxicity against the target tumor cells was determined in a calcein-release assay (Lichtenfels et al., 1994) by adding pre-activated PBMC to glioma target cells at an effector/target ratio of 1:100, 1:10 and 1:1. After 4 hours, fluorescence of supernatants was analyzed (Fluoroscan, Germany) and calculated as described (Lichtenfels et al., 1994). The allogenous cell cytotoxicity was enhanced by simultaneous treatment with the TGF-beta inhibitor, SD-208 as shown in FIG. 2.

### Example 4

In vivo effects of intratumoral application of TGF-beta type I receptor inhibitor, SD 208 with a combination of IL-2 and GM-CSF was investigated in a subcutaneous B16-F10 malignant mouse melanoma model (see FIG. 3). The subcutaneous model is widely used for the evaluation of therapy in many tumor models including B16 melanoma. Upon subcutaneous injection of 10⁵ cells/1004 PBS, B16 form a palpable tumor in 5-10 days and grow to a 1×1×1 cm tumor in 14-21 days, or upon subcutaneous injection of 10⁶ cells/1504 PBS, B16 form a palpable tumor in 8-9 days and grow to a 1×1×1 cm tumor in approx. 14 days

FIGS. 4A to 4C depict different models of intratumoral injection, wherein FIG. 4A refers to one intratumoral injection, FIG. 4B to two separate intratumoral injections and FIG. 4C to two separate intratumoral/intraparenchymal injections.

FIGS. 5A, 5B and 6 present two in vivo studies consisted of following experimental groups:

### Experiment 1) (FIG. 5A-5D)

1. Poloxamer 407+SD-208 (50 μg)+IL-2+GM-CSF
2. Control group (poloxamer 407)

### Experiment 2) (FIG. 6)

1. Poloxamer 407+SD-208+IL-2
2. Poloxamer 407+SD-208+IL-2+GM-CSF
3. Control group (poloxamer 407)
   each containing 8-10 animals after randomization.

On day 8 to 9, after a mean tumor volume of approx. 0.5-1.0 ml was reached, 45 tumor-bearing animals were randomized into treatment groups according to tumor sizes. On the same day (day 8/9), therapy has been initiated for all groups (Groups 1-2 and 1-3). Each tumor growth was recorded every second day by caliper measurement. On day 13/14 after last treatment the study was terminated, all animals sacrificed and a necropsy performed. At necropsy, animals were weighted and anaesthetized by isoflurane. Blood samples were collected from all animals via retroorbital vein puncture for preparation of serum, which was stored at −20° C. for further analysis (e.g., AST(GOT), ALT(GPT), AP, albumin, creatinine). After the blood sampling, animals were killed by cervical dislocation. Primary tumors were collected and wet weights (FIG. 5B) and volumes determined (FIG. 5A and FIG. 6). All primary tumors were snap-frozen in liquid nitrogen and stored appropriately at −80° C. In addition, the liver and kidney(s) were also collected and cryoconserved.

### Embodiments as Originally Filed

1. Combination of a TGF-beta inhibitor selected from the group consisting of an ALK-5 inhibitor, a neutralizing pan-anti-TGF-beta antibody, a neutralizing antibody against TGF-beta-1, or/and -2, or/and -3, a neutralizing anti-TGF-beta receptor type I or/and type II or/and type III antibody or binding fragments thereof, with IL-2 and GM-CSF for use in a method of treating a malignant tumor in a patient, wherein the TGF-beta inhibitor and the immune-stimulating cytokine(s) are administered intratumorally.

2. Combination for use in a method according to embodiment 1, wherein the combination is administered intratumorally into a tumor metastasis of a malignant tumor.

3. Combination for use in a method according to embodiment 1 or 2, wherein the combination further comprises an inhibitor of TGF-beta synthesis, selected from the group consisting of antisense oligonucleotides or/and siRNA or/and miRNA or/and antisense RNA, specific against mRNA encoding TGF-beta1, -2, and -3, isotypes.

4. Combination for use in a method according to any one of embodiments 1-3, wherein the combination comprises one or more additional immune stimulator(s) selected from the group consisting of IFNα, IFNβ, IFNγ, MIPα, MIPβ, TNFα, G-CSF, M-CSF, MIP, and interleukins (IL) like IL-1α, IL-1β, IL-3, IL-5, IL-6, IL-7, IL-12, IL-15, IL-16, IL-18, IL-21, IL-24, IL-32, IL-33, IL-36.

5. Combination for use in a method according to any one of embodiments 1-3, wherein the combination comprises one or more additional immune stimulator(s) selected from the group consisting of ligands binding to receptors like CD40, CD134 (OX40) and CD137, TIM-3, immunoglobulin-like transcript (ILT), glucocorticoid-induced tumor necrosis factor family receptor (GITR), and/or inhibitors of prostaglandin E (PGE), interleukin 10 (IL-10), interleukin 35 (IL-35), and/or melanoma inhibiting activity (MIA).

6. Combination for use in a method according to any one of embodiments 1-3, wherein the combination comprises one or more additional immune modulators(s) selected from the group consisting of inhibitors of immune-checkpoints like cytotoxic T-lymphocyte antigen 4 (CTLA-4), programmed cell death protein 1 (PD-1), or other molecules binding PD-L1 and/or PD-L2.

7. Combination for use in a method according to any one of embodiments 1-6, wherein the TGF-beta inhibitor, and the immune stimulator are administered at the same time or subsequently.

8. Combination for use in a method according to any one of embodiments 1-7, wherein the intratumoral concentration of the ALK5 inhibitor is in the range of about 0.1 μM to about 1 mM.

9. Combination for use in a method according to any one of embodiments 1-8, wherein the intratumoral concentration of the immune stimulator IL-2 is in a range of 1.8×10⁶ to 18×10⁶ (IU) and/or the intratumoral concentration of the immune stimulator GM-CSF is in a range of 0.1-0.5 mg.

10. Pharmaceutical composition comprising a combination according to any one of embodiments 1 to 9 and a pharmaceutically acceptable carrier for use in a method of treating a malignant tumor in a patient.

11. Pharmaceutical composition for use in a method according to embodiment 10, wherein the composition is administered intratumorally.

12. Pharmaceutical composition for use in a method according to embodiment 10 or 11, wherein the pharmaceutically acceptable carrier is a poloxamer selected from the group consisting of poloxamer 124, 188, 237, 338, 388 and 407.

1. A method of treating a malignant tumor in a patient, the method comprising administering a combination of a TGF-beta inhibitor selected from the group consisting of an ALK-5 inhibitor, a neutralizing pan-anti-TGF-beta antibody, a neutralizing antibody against TGF-beta-1, or/and -2, or/and -3, a neutralizing anti-TGF-beta receptor type I or/and type II or/and type III antibody or binding fragments thereof, with IL-2 and GM-CSF, wherein the TGF-beta inhibitor and the immune-stimulating cytokine(s) are administered intratumorally.

2. The method according to claim 1, wherein the combination is administered intratumorally into a tumor metastasis of a malignant tumor.

3. The method according to claim 1, wherein the combination further comprises an inhibitor of TGF-beta synthesis, selected from the group consisting of antisense oligonucleotides or/and siRNA or/and miRNA or/and antisense RNA, specific against mRNA encoding TGF-beta1, -2, and -3, isotypes.

4. The method according to claim 1, wherein the combination comprises one or more additional immune stimulator(s) selected from the group consisting of IFNα, IFNβ, IFNγ, MIPα, MIPβ, TNFα, G-CSF, M-CSF, MIP, and interleukins (IL) like IL-1α, IL-1β, IL-3, IL-5, IL-6, IL-7, IL-12, IL-15, IL-16, IL-18, IL-21, IL-24, IL-32, IL-33, IL-36.

5. The method according to claim 1, wherein the combination comprises one or more additional immune stimulator(s) selected from the group consisting of ligands binding to receptors like CD40, CD134 (OX40) and CD137, TIM-3, immunoglobulin-like transcript (ILT), glucocorticoid-induced tumor necrosis factor family receptor (GITR), and/or inhibitors of prostaglandin E (PGE), interleukin 10 (IL-10), interleukin 35 (IL-35), and/or melanoma inhibiting activity (MIA).

6. The method according to claim 1, wherein the combination comprises one or more additional immune modulators(s) selected from the group consisting of inhibitors of immune-checkpoints like cytotoxic T-lymphocyte antigen 4 (CTLA-4), programmed cell death protein 1 (PD-1), or other molecules binding PD-L1 and/or PD-L2.

7. The method according to claim 1, wherein the TGF-beta inhibitor, and the immune stimulator are administered at the same time or subsequently.

8. The method according to claim 1, wherein the intratumoral concentration of the ALK5 inhibitor is in the range of about 0.1 μM to about 1 mM.

9. The method according to claim 1, wherein the intratumoral concentration of the immune stimulator IL-2 is in a range of 1.8×10⁶ to 18×10⁶ (IU) and/or the intratumoral concentration of the immune stimulator GM-CSF is in a range of 0.1-0.5 mg.

10. A method of treating a malignant tumor in a patient comprising administering a pharmaceutical composition comprising a combination of a TGF-beta inhibitor selected from the group consisting of an ALK-5 inhibitor, a neutralizing pan-anti-TGF-beta antibody, a neutralizing antibody against TGF-beta-1, or/and -2, or/and -3, a neutralizing anti-TGF-beta receptor type I or/and type II or/and type III antibody or binding fragments thereof, with IL-2 and GM-CSF, and a pharmaceutically acceptable carrier.

11. The method according to claim 10, wherein the composition is administered intratumorally.

12. The method according to claim 10, wherein the pharmaceutically acceptable carrier is a poloxamer selected from the group consisting of poloxamer 124, 188, 237, 338, 388 and 407.


SEQUENCE LISTING (392 sequences)

SEQ ID NO: 1 - protein, 10 aa, Artificial Sequence
  Tumor Antigens Resulting from Mutations
FIASNGVKLV

SEQ ID NO: 2 - protein, 16 aa, Artificial Sequence
  Tumor Antigens Resulting from Mutations
YSVYFNLPADTIYTNH

SEQ ID NO: 3 - protein, 9 aa, Artificial Sequence
  Tumor Antigens Resulting from Mutations
SSKALQRPV

SEQ ID NO: 4 - protein, 9 aa, Artificial Sequence
  Tumor Antigens Resulting from Mutations
GFKQSSKAL

SEQ ID NO: 5 - protein, 17 aa, Artificial Sequence
  Tumor Antigens Resulting from Mutations
ATGFKQSSKALQRPVAS

SEQ ID NO: 6 - protein, 29 aa, Artificial Sequence
  Tumor Antigens Resulting from Mutations
EDLTVKIGDFGLATEKSRWSGSHQFEQLS

SEQ ID NO: 7 - protein, 9 aa, Artificial Sequence
  Tumor Antigens Resulting from Mutations
FLIIWQNTM

SEQ ID NO: 8 - protein, 9 aa, Artificial Sequence
  Tumor Antigens Resulting from Mutations
FPSDSWCYF

SEQ ID NO: 9 - protein, 9 aa, Artificial Sequence
  Tumor Antigens Resulting from Mutations
SYLDSGIHF

SEQ ID NO: 10 - protein, 12 aa, Artificial Sequence
  Tumor Antigens Resulting from Mutations
FSWAMDLDPKGA

SEQ ID NO: 11 - protein, 10 aa, Artificial Sequence
  Tumor Antigens Resulting from Mutations
ACDPHSGHFV

SEQ ID NO: 12 - protein, 9 aa, Artificial Sequence
  Tumor Antigens Resulting from Mutations
AVCPWTWLR

SEQ ID NO: 13 - protein, 9 aa, Artificial Sequence
  Tumor Antigens Resulting from Mutations
ILDKVLVHL

SEQ ID NO: 14 - protein, 14 aa, Artificial Sequence
  Tumor Antigens Resulting from Mutations
TLYQDDTLTLQAAG

SEQ ID NO: 15 - protein, 16 aa, Artificial Sequence
  Tumor Antigens Resulting from Mutations
TMKQICKKEIRRLHQY

SEQ ID NO: 16 - protein, 10 aa, Artificial Sequence
  Tumor Antigens Resulting from Mutations
KILDAVVAQK

SEQ ID NO: 17 - protein, 9 aa, Artificial Sequence
  Tumor Antigens Resulting from Mutations
ETVSEQSNV

SEQ ID NO: 18 - protein, 10 aa, Artificial Sequence
  Tumor Antigens Resulting from Mutations
RIAECILGMI

SEQ ID NO: 19 - protein, 15 aa, Artificial Sequence
  Tumor Antigens Resulting from Mutations
IGRIAECILGMNPSR

SEQ ID NO: 20 - protein, 10 aa, Artificial Sequence
  Tumor Antigens Resulting from Mutations
YVDFREYEYY

SEQ ID NO: 21 - protein, 14 aa, Artificial Sequence
  Tumor Antigens Resulting from Mutations
MIFEKHGFRRTTPP

SEQ ID NO: 22 - protein, 10 aa, Artificial Sequence
  Tumor Antigens Resulting from Mutations
TLDWLLQTPK

SEQ ID NO: 23 - protein, 9 aa, Artificial Sequence
  Tumor Antigens Resulting from Mutations
WRRAPAPGA

SEQ ID NO: 24 - protein, 9 aa, Artificial Sequence
  Tumor Antigens Resulting from Mutations
PVTWRRAPA

SEQ ID NO: 25 - protein, 10 aa, Artificial Sequence
  Tumor Antigens Resulting from Mutations
SLFEGIDIYT

SEQ ID NO: 26 - protein, 9 aa, Artificial Sequence
  Tumor Antigens Resulting from Mutations
AEPINIQTW

SEQ ID NO: 27 - protein, 11 aa, Artificial Sequence
  Tumor Antigens Resulting from Mutations
FLEGNEVGKTY

SEQ ID NO: 28 - protein, 9 aa, Artificial Sequence
  Tumor Antigens Resulting from Mutations
FLDEFMEGV

SEQ ID NO: 29 - protein, 9 aa, Artificial Sequence
  Tumor Antigens Resulting from Mutations
EEKLIVVLF

SEQ ID NO: 30 - protein, 11 aa, Artificial Sequence
  Tumor Antigens Resulting from Mutations
SELFRSGLDSY

SEQ ID NO: 31 - protein, 9 aa, Artificial Sequence
  Tumor Antigens Resulting from Mutations
EAFIQPITR

SEQ ID NO: 32 - protein, 11 aa, Artificial Sequence
  Tumor Antigens Resulting from Mutations
RVIKNSIRLTL

SEQ ID NO: 33 - protein, 9 aa, Artificial Sequence
  Tumor Antigens Resulting from Mutations
KINKNPKYK

SEQ ID NO: 34 - protein, 8 aa, Artificial Sequence
  Tumor Antigens Resulting from Mutations
QQITKTEV

SEQ ID NO: 35 - protein, 10 aa, Artificial Sequence
  Tumor Antigens Resulting from Mutations
SLYKFSPFPL

SEQ ID NO: 36 - protein, 9 aa, Artificial Sequence
  Tumor Antigens Resulting from Mutations
KELEGILLL

SEQ ID NO: 37 - protein, 9 aa, Artificial Sequence
  Tumor Antigens Resulting from Mutations
VVPCEPPEV

SEQ ID NO: 38 - protein, 25 aa, Artificial Sequence
  Tumor Antigens Resulting from Mutations
NSNHVASGAGEAAIETQSSSSEEIV

SEQ ID NO: 39 - protein, 10 aa, Artificial Sequence
  Tumor Antigens Resulting from Mutations
LLLDDLLVSI

SEQ ID NO: 40 - protein, 16 aa, Artificial Sequence
  Tumor Antigens Resulting from Mutations
PYYFAAELPPRNLPEP

SEQ ID NO: 41 - protein, 9 aa, Artificial Sequence
  Tumor Antigens Resulting from Mutations
VVVGAVGVG

SEQ ID NO: 42 - protein, 10 aa, Artificial Sequence
  Tumor Antigens Resulting from Mutations
ILDTAGREEY

SEQ ID NO: 43 - protein, 9 aa, Artificial Sequence
  Tumor Antigens Resulting from Mutations
RPHVPESAF

SEQ ID NO: 44 - protein, 9 aa, Artificial Sequence
  Tumor Antigens Resulting from Mutations
KIFSEVTLK

SEQ ID NO: 45 - protein, 9 aa, Artificial Sequence
  Tumor Antigens Resulting from Mutations
SHETVIIEL

SEQ ID NO: 46 - protein, 10 aa, Artificial Sequence
  Tumor Antigens Resulting from Mutations
QRPYGYDQIM

SEQ ID NO: 47 - protein, 9 aa, Artificial Sequence
  Tumor Antigens Resulting from Mutations
RLSSCVPVA

SEQ ID NO: 48 - protein, 15 aa, Artificial Sequence
  Tumor Antigens Resulting from Mutations
GELIGILNAAKVPAD

SEQ ID NO: 49 - protein, 9 aa, Artificial Sequence
  Shared Tumor-Specific Antigens
AARAVFLAL

SEQ ID NO: 50 - protein, 11 aa, Artificial Sequence
  Shared Tumor-Specific Antigens
SLIAAAAFCLA

SEQ ID NO: 51 - protein, 9 aa, Artificial Sequence
  Shared Tumor-Specific Antigens
FLDRFLSCM

SEQ ID NO: 52 - protein, 8 aa, Artificial Sequence
  Shared Tumor-Specific Antigens
YRPRPRRY

SEQ ID NO: 53 - protein, 9 aa, Artificial Sequence
  Shared Tumor-Specific Antigens
YYWPRPRRY

SEQ ID NO: 54 - protein, 10 aa, Artificial Sequence
  Shared Tumor-Specific Antigens
VLPDVFIRCV

SEQ ID NO: 55 - protein, 9 aa, Artificial Sequence
  Shared Tumor-Specific Antigens
VLPDVFIRC

SEQ ID NO: 56 - protein, 9 aa, Artificial Sequence
  Shared Tumor-Specific Antigens
MLAVISCAV

SEQ ID NO: 57 - protein, 9 aa, Artificial Sequence
  Shared Tumor-Specific Antigens
RQKRILVNL

SEQ ID NO: 58 - protein, 9 aa, Artificial Sequence
  Shared Tumor-Specific Antigens
NYNNFYRFL

SEQ ID NO: 59 - protein, 9 aa, Artificial Sequence
  Shared Tumor-Specific Antigens
EYLSLSDKI

SEQ ID NO: 60 - protein, 10 aa, Artificial Sequence
  Shared Tumor-Specific Antigens
EYSKECLKEF

SEQ ID NO: 61 - protein, 11 aa, Artificial Sequence
  Shared Tumor-Specific Antigens
MLMAQEALAFL

SEQ ID NO: 62 - protein, 9 aa, Artificial Sequence
  Shared Tumor-Specific Antigens
SLLMWITQC

SEQ ID NO: 63 - protein, 11 aa, Artificial Sequence
  Shared Tumor-Specific Antigens
MLMAQEALAFL

SEQ ID NO: 64 - protein, 10 aa, Artificial Sequence
  Shared Tumor-Specific Antigens
LAAQERRVPR

SEQ ID NO: 65 - protein, 10 aa, Artificial Sequence
  Shared Tumor-Specific Antigens
LELVRRILSR

SEQ ID NO: 66 - protein, 9 aa, Artificial Sequence
  Shared Tumor-Specific Antigens
APRGVRMAV

SEQ ID NO: 67 - protein, 20 aa, Artificial Sequence
  Shared Tumor-Specific Antigens
QGAMLAAQERRVPRAAEVPR

SEQ ID NO: 68 - protein, 13 aa, Artificial Sequence
  Shared Tumor-Specific Antigens
ILSRDAAPLPRPG

SEQ ID NO: 69 - protein, 18 aa, Artificial Sequence
  Shared Tumor-Specific Antigens
AADHRQLQLSISSCLQQL

SEQ ID NO: 70 - protein, 14 aa, Artificial Sequence
  Shared Tumor-Specific Antigens
SLLMWITQCFLPVF

SEQ ID NO: 71 - protein, 22 aa, Artificial Sequence
  Shared Tumor-Specific Antigens
CLSRRPWKRSWSAGSCPGMPHL

SEQ ID NO: 72 - protein, 14 aa, Artificial Sequence
  Shared Tumor-Specific Antigens
AGATGGRGPRGAGA

SEQ ID NO: 73 - protein, 14 aa, Artificial Sequence
  Shared Tumor-Specific Antigens
AGATGGRGPRGAGA

SEQ ID NO: 74 - protein, 9 aa, Artificial Sequence
  Shared Tumor-Specific Antigens
EADPTGHSY

SEQ ID NO: 75 - protein, 9 aa, Artificial Sequence
  Shared Tumor-Specific Antigens
KVLEYVIKV

SEQ ID NO: 76 - protein, 9 aa, Artificial Sequence
  Shared Tumor-Specific Antigens
SLFRAVITK

SEQ ID NO: 77 - protein, 10 aa, Artificial Sequence
  Shared Tumor-Specific Antigens
EVYDGREHSA

SEQ ID NO: 78 - protein, 9 aa, Artificial Sequence
  Shared Tumor-Specific Antigens
RVRFFFPSL

SEQ ID NO: 79 - protein, 9 aa, Artificial Sequence
  Shared Tumor-Specific Antigens
EADPTGHSY

SEQ ID NO: 80 - protein, 10 aa, Artificial Sequence
  Shared Tumor-Specific Antigens
REPVTKAEML

SEQ ID NO: 81 - protein, 10 aa, Artificial Sequence
  Shared Tumor-Specific Antigens
KEADPTGHSY

SEQ ID NO: 82 - protein, 9 aa, Artificial Sequence
  Shared Tumor-Specific Antigens
SAFPTTINF

SEQ ID NO: 83 - protein, 9 aa, Artificial Sequence
  Shared Tumor-Specific Antigens
DPARYEFLW

SEQ ID NO: 84 - protein, 11 aa, Artificial Sequence
  Shared Tumor-Specific Antigens
ITKKVADLVGF

SEQ ID NO: 85 - protein, 9 aa, Artificial Sequence
  Shared Tumor-Specific Antigens
SAYGEPRKL

SEQ ID NO: 86 - protein, 9 aa, Artificial Sequence
  Shared Tumor-Specific Antigens
RVRFFFPSL

SEQ ID NO: 87 - protein, 9 aa, Artificial Sequence
  Shared Tumor-Specific Antigens
SAYGEPRKL

SEQ ID NO: 88 - protein, 15 aa, Artificial Sequence
  Shared Tumor-Specific Antigens
TSCILESLFRAVITK

SEQ ID NO: 89 - protein, 9 aa, Artificial Sequence
  Shared Tumor-Specific Antigens
EYLQLVFGI

SEQ ID NO: 90 - protein, 15 aa, Artificial Sequence
  Shared Tumor-Specific Antigens
TSCILESLFRAVITK

SEQ ID NO: 91 - protein, 15 aa, Artificial Sequence
  Shared Tumor-Specific Antigens
PRALAETSYVKVLEY

SEQ ID NO: 92 - protein, 10 aa, Artificial Sequence
  Shared Tumor-Specific Antigens
YLQLVFGIEV

SEQ ID NO: 93 - protein, 16 aa, Artificial Sequence
  Shared Tumor-Specific Antigens
FLLLKYRAREPVTKAE

SEQ ID NO: 94 - protein, 12 aa, Artificial Sequence
  Shared Tumor-Specific Antigens
EYVIKVSARVRF

SEQ ID NO: 95 - protein, 12 aa, Artificial Sequence
  Shared Tumor-Specific Antigens
EYVIKVSARVRF

SEQ ID NO: 96 - protein, 12 aa, Artificial Sequence
  Shared Tumor-Specific Antigens
EYVIKVSARVRF

SEQ ID NO: 97 - protein, 9 aa, Artificial Sequence
  Shared Tumor-Specific Antigens
EGDCAPEEK

SEQ ID NO: 98 - protein, 9 aa, Artificial Sequence
  Shared Tumor-Specific Antigens
EYLQLVFGI

SEQ ID NO: 99 - protein, 14 aa, Artificial Sequence
  Shared Tumor-Specific Antigens
LLKYRAREPVTKAE

SEQ ID NO: 100 - protein, 9 aa, Artificial Sequence
  Shared Tumor-Specific Antigens
EVDPIGHLY

SEQ ID NO: 101 - protein, 9 aa, Artificial Sequence
  Shared Tumor-Specific Antigens
FLWGPRALV

SEQ ID NO: 102 - protein, 9 aa, Artificial Sequence
  Shared Tumor-Specific Antigens
KVAELVHFL

SEQ ID NO: 103 - protein, 9 aa, Artificial Sequence
  Shared Tumor-Specific Antigens
TFPDLESEF

SEQ ID NO: 104 - protein, 9 aa, Artificial Sequence
  Shared Tumor-Specific Antigens
VAELVHFLL

SEQ ID NO: 105 - protein, 10 aa, Artificial Sequence
  Shared Tumor-Specific Antigens
MEVDPIGHLY

SEQ ID NO: 106 - protein, 9 aa, Artificial Sequence
  Shared Tumor-Specific Antigens
EVDPIGHLY

SEQ ID NO: 107 - protein, 10 aa, Artificial Sequence
  Shared Tumor-Specific Antigens
MEVDPIGHLY

SEQ ID NO: 108 - protein, 10 aa, Artificial Sequence
  Shared Tumor-Specific Antigens
REPVTKAEML

SEQ ID NO: 109 - protein, 9 aa, Artificial Sequence
  Shared Tumor-Specific Antigens
AELVHFLLL

SEQ ID NO: 110 - protein, 9 aa, Artificial Sequence
  Shared Tumor-Specific Antigens
WQYFFPVIF

SEQ ID NO: 111 - protein, 9 aa, Artificial Sequence
  Shared Tumor-Specific Antigens
EGDCAPEEK

SEQ ID NO: 112 - protein, 16 aa, Artificial Sequence
  Shared Tumor-Specific Antigens
KKLLTQHFVQENYLEY

SEQ ID NO: 113 - protein, 15 aa, Artificial Sequence
  Shared Tumor-Specific Antigens
RKVAELVHFLLLKYR

SEQ ID NO: 114 - protein, 12 aa, Artificial Sequence
  Shared Tumor-Specific Antigens
VIFSKASSSLQL

SEQ ID NO: 115 - protein, 16 aa, Artificial Sequence
  Shared Tumor-Specific Antigens
KKLLTQHFVQENYLEY

SEQ ID NO: 116 - protein, 16 aa, Artificial Sequence
  Shared Tumor-Specific Antigens
ACYEFLWGPRALVETS

SEQ ID NO: 117 - protein, 15 aa, Artificial Sequence
  Shared Tumor-Specific Antigens
RKVAELVHFLLLKYR

SEQ ID NO: 118 - protein, 12 aa, Artificial Sequence
  Shared Tumor-Specific Antigens
VIFSKASSSLQL

SEQ ID NO: 119 - protein, 16 aa, Artificial Sequence
  Shared Tumor-Specific Antigens
FLLLKYRAREPVTKAE

SEQ ID NO: 120 - protein, 15 aa, Artificial Sequence
  Shared Tumor-Specific Antigens
VFGIELMEVDPIGHL

SEQ ID NO: 121 - protein, 15 aa, Artificial Sequence
  Shared Tumor-Specific Antigens
GDNQIMPKAGLLIIV

SEQ ID NO: 122 - protein, 15 aa, Artificial Sequence
  Shared Tumor-Specific Antigens
TSYVKVLHHMVKISG

SEQ ID NO: 123 - protein, 16 aa, Artificial Sequence
  Shared Tumor-Specific Antigens
RKVAELVHFLLLKYRA

SEQ ID NO: 124 - protein, 9 aa, Artificial Sequence
  Shared Tumor-Specific Antigens
EVDPASNTY

SEQ ID NO: 125 - protein, 10 aa, Artificial Sequence
  Shared Tumor-Specific Antigens
GVYDGREHTV

SEQ ID NO: 126 - protein, 9 aa, Artificial Sequence
  Shared Tumor-Specific Antigens
NYKRCFPVI

SEQ ID NO: 127 - protein, 8 aa, Artificial Sequence
  Shared Tumor-Specific Antigens
SESLKMIF

SEQ ID NO: 128 - protein, 9 aa, Artificial Sequence
  Shared Tumor-Specific Antigens
EVDPIGHVY

SEQ ID NO: 129 - protein, 10 aa, Artificial Sequence
  Shared Tumor-Specific Antigens
REPVTKAEML

SEQ ID NO: 130 - protein, 9 aa, Artificial Sequence
  Shared Tumor-Specific Antigens
EGDCAPEEK

SEQ ID NO: 131 - protein, 9 aa, Artificial Sequence
  Shared Tumor-Specific Antigens
ISGGPRISY

SEQ ID NO: 132 - protein, 14 aa, Artificial Sequence
  Shared Tumor-Specific Antigens
LLKYRAREPVTKAE

SEQ ID NO: 133 - protein, 9 aa, Artificial Sequence
  Shared Tumor-Specific Antigens
ALSVMGVYV

SEQ ID NO: 134 - protein, 10 aa, Artificial Sequence
  Shared Tumor-Specific Antigens
GLYDGMEHLL

SEQ ID NO: 135 - protein, 9 aa, Artificial Sequence
  Shared Tumor-Specific Antigens
DPARYEFLW

SEQ ID NO: 136 - protein, 9 aa, Artificial Sequence
  Shared Tumor-Specific Antigens
EGDCAPEEK

SEQ ID NO: 137 - protein, 15 aa, Artificial Sequence
  Shared Tumor-Specific Antigens
REPFTKAEMLGSVIR

SEQ ID NO: 138 - protein, 9 aa, Artificial Sequence
  Shared Tumor-Specific Antigens
VRIGHLYIL

SEQ ID NO: 139 - protein, 9 aa, Artificial Sequence
  Shared Tumor-Specific Antigens
FLWGPRALV

SEQ ID NO: 140 - protein, 14 aa, Artificial Sequence
  Shared Tumor-Specific Antigens
AELVHFLLLKYRAR

SEQ ID NO: 141 - protein, 13 aa, Artificial Sequence
  Shared Tumor-Specific Antigens
SSALLSIFQSSPE

SEQ ID NO: 142 - protein, 10 aa, Artificial Sequence
  Shared Tumor-Specific Antigens
ILFGISLREV

SEQ ID NO: 143 - protein, 9 aa, Artificial Sequence
  Shared Tumor-Specific Antigens
SFSYTLLSL

SEQ ID NO: 144 - protein, 9 aa, Artificial Sequence
  Shared Tumor-Specific Antigens
VSSFFSYTL

SEQ ID NO: 145 - protein, 9 aa, Artificial Sequence
  Shared Tumor-Specific Antigens
KVVEFLAML

SEQ ID NO: 146 - protein, 10 aa, Artificial Sequence
  Shared Tumor-Specific Antigens
LLFGLALIEV

SEQ ID NO: 147 - protein, 15 aa, Artificial Sequence
  Shared Tumor-Specific Antigens
SSTLYLVFSPSSFST

SEQ ID NO: 148 - protein, 9 aa, Artificial Sequence
  Shared Tumor-Specific Antigens
ASSTLYLVF

SEQ ID NO: 149 - protein, 9 aa, Artificial Sequence
  Shared Tumor-Specific Antigens
SESIKKKVL

SEQ ID NO: 150 - protein, 9 aa, Artificial Sequence
  Shared Tumor-Specific Antigens
ALKDVEERV

SEQ ID NO: 151 - protein, 20 aa, Artificial Sequence
  Shared Tumor-Specific Antigens
PDTRPAPGSTAPPAHGVTSA

SEQ ID NO: 152 - protein, 9 aa, Artificial Sequence
  Shared Tumor-Specific Antigens
QGQHFLQKV

SEQ ID NO: 153 - protein, 10 aa, Artificial Sequence
  Shared Tumor-Specific Antigens
LAAQERRVPR

SEQ ID NO: 154 - protein, 10 aa, Artificial Sequence
  Shared Tumor-Specific Antigens
TVSGNILTIR

SEQ ID NO: 155 - protein, 13 aa, Artificial Sequence
  Shared Tumor-Specific Antigens
APRGPHGGAASGL

SEQ ID NO: 156 - protein, 9 aa, Artificial Sequence
  Shared Tumor-Specific Antigens
MPFATPMEA

SEQ ID NO: 157 - protein, 12 aa, Artificial Sequence
  Shared Tumor-Specific Antigens
KEFTVSGNILTI

SEQ ID NO: 158 - protein, 9 aa, Artificial Sequence
  Shared Tumor-Specific Antigens
LAMPFATPM

SEQ ID NO: 159 - protein, 11 aa, Artificial Sequence
  Shared Tumor-Specific Antigens
MLMAQEALAFL

SEQ ID NO: 160 - protein, 9 aa, Artificial Sequence
  Shared Tumor-Specific Antigens
ARGPESRLL

SEQ ID NO: 161 - protein, 14 aa, Artificial Sequence
  Shared Tumor-Specific Antigens
SLLMWITQCFLPVF

SEQ ID NO: 162 - protein, 9 aa, Artificial Sequence
  Shared Tumor-Specific Antigens
SLLMWITQC

SEQ ID NO: 163 - protein, 10 aa, Artificial Sequence
  Shared Tumor-Specific Antigens
ASGPGGGAPR

SEQ ID NO: 164 - protein, 25 aa, Artificial Sequence
  Shared Tumor-Specific Antigens
LLEFYLAMPFATPMEAELARRSLAQ

SEQ ID NO: 165 - protein, 12 aa, Artificial Sequence
  Shared Tumor-Specific Antigens
RLLEFYLAMPFA

SEQ ID NO: 166 - protein, 25 aa, Artificial Sequence
  Shared Tumor-Specific Antigens
PGVLLKEFTVSGNILTIRLTAADHR

SEQ ID NO: 167 - protein, 13 aa, Artificial Sequence
  Shared Tumor-Specific Antigens
PFATPMEAELARR

SEQ ID NO: 168 - protein, 20 aa, Artificial Sequence
  Shared Tumor-Specific Antigens
QGAMLAAQERRVPRAAEVPR

SEQ ID NO: 169 - protein, 20 aa, Artificial Sequence
  Shared Tumor-Specific Antigens
PGVLLKEFTVSGNILTIRLT

SEQ ID NO: 170 - protein, 10 aa, Artificial Sequence
  Shared Tumor-Specific Antigens
VLLKEFTVSG

SEQ ID NO: 171 - protein, 18 aa, Artificial Sequence
  Shared Tumor-Specific Antigens
AADHRQLQLSISSCLQQL

SEQ ID NO: 172 - protein, 11 aa, Artificial Sequence
  Shared Tumor-Specific Antigens
KEFTVSGNILT

SEQ ID NO: 173 - protein, 15 aa, Artificial Sequence
  Shared Tumor-Specific Antigens
LKEFTVSGNILTIRL

SEQ ID NO: 174 - protein, 14 aa, Artificial Sequence
  Shared Tumor-Specific Antigens
LLEFYLAMPFATPM

SEQ ID NO: 175 - protein, 14 aa, Artificial Sequence
  Shared Tumor-Specific Antigens
AGATGGRGPRGAGA

SEQ ID NO: 176 - protein, 9 aa, Artificial Sequence
  Shared Tumor-Specific Antigens
LYATVIHDI

SEQ ID NO: 177 - protein, 9 aa, Artificial Sequence
  Shared Tumor-Specific Antigens
ILDSSEEDK

SEQ ID NO: 178 - protein, 9 aa, Artificial Sequence
  Shared Tumor-Specific Antigens
KASEKIFYV

SEQ ID NO: 179 - protein, 16 aa, Artificial Sequence
  Shared Tumor-Specific Antigens
EKIQKAFDDIAKYFSK

SEQ ID NO: 180 - protein, 11 aa, Artificial Sequence
  Shared Tumor-Specific Antigens
FGRLQGISPKI

SEQ ID NO: 181 - protein, 18 aa, Artificial Sequence
  Shared Tumor-Specific Antigens
WEKMKASEKIFYVYMKRK

SEQ ID NO: 182 - protein, 15 aa, Artificial Sequence
  Shared Tumor-Specific Antigens
KIFYVYMKRKYEAMT

SEQ ID NO: 183 - protein, 20 aa, Artificial Sequence
  Shared Tumor-Specific Antigens
INKTSGPKRGKHAWTHRLRE

SEQ ID NO: 184 - protein, 20 aa, Artificial Sequence
  Shared Tumor-Specific Antigens
KHAWTHRLRERKQLVVYEEI

SEQ ID NO: 185 - protein, 20 aa, Artificial Sequence
  Shared Tumor-Specific Antigens
YFSKKEWEKMKSSEKIVYVY

SEQ ID NO: 186 - protein, 20 aa, Artificial Sequence
  Shared Tumor-Specific Antigens
KSSEKIVYVYMKLNYEVMTK

SEQ ID NO: 187 - protein, 20 aa, Artificial Sequence
  Shared Tumor-Specific Antigens
MKLNYEVMTKLGFKVTLPPF

SEQ ID NO: 188 - protein, 20 aa, Artificial Sequence
  Shared Tumor-Specific Antigens
LGFKVTLPPFMRSKRAADFH

SEQ ID NO: 189 - protein, 20 aa, Artificial Sequence
  Shared Tumor-Specific Antigens
KHAWTHRLRERKQLVVYEEI

SEQ ID NO: 190 - protein, 9 aa, Artificial Sequence
  Shared Tumor-Specific Antigens
SLGWLFLLL

SEQ ID NO: 191 - protein, 9 aa, Artificial Sequence
  Shared Tumor-Specific Antigens
LSRLSNRLL

SEQ ID NO: 192 - protein, 9 aa, Artificial Sequence
  Shared Tumor-Specific Antigens
LSRLSNRLL

SEQ ID NO: 193 - protein, 15 aa, Artificial Sequence
  Shared Tumor-Specific Antigens
CEFHACWPAFTVLGE

SEQ ID NO: 194 - protein, 10 aa, Artificial Sequence
  Shared Tumor-Specific Antigens
EVISCKLIKR

SEQ ID NO: 195 - protein, 9 aa, Artificial Sequence
  Shared Tumor-Specific Antigens
RQKKIRIQL

SEQ ID NO: 196 - protein, 16 aa, Artificial Sequence
  Shared Tumor-Specific Antigens
HLGSRQKKIRIQLRSQ

SEQ ID NO: 197 - protein, 17 aa, Artificial Sequence
  Shared Tumor-Specific Antigens
CATWKVICKSCISQTPG

SEQ ID NO: 198 - protein, 9 aa, Artificial Sequence
  Differentiation Antigens
YLSGANLNL

SEQ ID NO: 199 - protein, 9 aa, Artificial Sequence
  Differentiation Antigens
IMIGVLVGV

SEQ ID NO: 200 - protein, 9 aa, Artificial Sequence
  Differentiation Antigens
GVLVGVALI

SEQ ID NO: 201 - protein, 9 aa, Artificial Sequence
  Differentiation Antigens
HLFGYSWYK

SEQ ID NO: 202 - protein, 25 aa, Artificial Sequence
  Differentiation Antigens
DTGFYTLHVIKSDLVNEEATGQFRV

SEQ ID NO: 203 - protein, 15 aa, Artificial Sequence
  Differentiation Antigens
YSWRINGIPQQHTQV

SEQ ID NO: 204 - protein, 10 aa, Artificial Sequence
  Differentiation Antigens
QYSWFVNGTF

SEQ ID NO: 205 - protein, 13 aa, Artificial Sequence
  Differentiation Antigens
TYYRPGVNLSLSC

SEQ ID NO: 206 - protein, 13 aa, Artificial Sequence
  Differentiation Antigens
EIIYPNASLLIQN

SEQ ID NO: 207 - protein, 9 aa, Artificial Sequence
  Differentiation Antigens
TYACFVSNL

SEQ ID NO: 208 - protein, 15 aa, Artificial Sequence
  Differentiation Antigens
YACFVSNLATGRNNS

SEQ ID NO: 209 - protein, 13 aa, Artificial Sequence
  Differentiation Antigens
LWWVNNQSLPVSP

SEQ ID NO: 210 - protein, 15 aa, Artificial Sequence
  Differentiation Antigens
AYVCGIQNSVSANRS

SEQ ID NO: 211 - protein, 13 aa, Artificial Sequence
  Differentiation Antigens
EIIYPNASLLIQN

SEQ ID NO: 212 - protein, 13 aa, Artificial Sequence
  Differentiation Antigens
NSIVKSITVSASG

SEQ ID NO: 213 - protein, 9 aa, Artificial Sequence
  Differentiation Antigens
KTWGQYWQV

SEQ ID NO: 214 - protein, 10 aa, Artificial Sequence
  Differentiation Antigens
AMLGTHTMEV

SEQ ID NO: 215 - protein, 9 aa, Artificial Sequence
  Differentiation Antigens
MLGTHTMEV

SEQ ID NO: 216 - protein, 9 aa, Artificial Sequence
  Differentiation Antigens
ITDQVPFSV

SEQ ID NO: 217 - protein, 9 aa, Artificial Sequence
  Differentiation Antigens
YLEPGPVTA

SEQ ID NO: 218 - protein, 10 aa, Artificial Sequence
  Differentiation Antigens
LLDGTATLRL

SEQ ID NO: 219 - protein, 10 aa, Artificial Sequence
  Differentiation Antigens
VLYRYGSFSV

SEQ ID NO: 220 - protein, 10 aa, Artificial Sequence
  Differentiation Antigens
SLADTNSLAV

SEQ ID NO: 221 - protein, 9 aa, Artificial Sequence
  Differentiation Antigens
RLMKQDFSV

SEQ ID NO: 222 - protein, 9 aa, Artificial Sequence
  Differentiation Antigens
RLPRIFCSC

SEQ ID NO: 223 - protein, 9 aa, Artificial Sequence
  Differentiation Antigens
LIYRRRLMK

SEQ ID NO: 224 - protein, 9 aa, Artificial Sequence
  Differentiation Antigens
ALLAVGATK

SEQ ID NO: 225 - protein, 10 aa, Artificial Sequence
  Differentiation Antigens
IALNFPGSQK

SEQ ID NO: 226 - protein, 9 aa, Artificial Sequence
  Differentiation Antigens
ALNFPGSQK

SEQ ID NO: 227 - protein, 9 aa, Artificial Sequence
  Differentiation Antigens
VYFFLPDHL

SEQ ID NO: 228 - protein, 9 aa, Artificial Sequence
  Differentiation Antigens
RTKQLYPEW

SEQ ID NO: 229 - protein, 10 aa, Artificial Sequence
  Differentiation Antigens
HTMEVTVYHR

SEQ ID NO: 230 - protein, 9 aa, Artificial Sequence
  Differentiation Antigens
SSPGCQPPA

SEQ ID NO: 231 - protein, 10 aa, Artificial Sequence
  Differentiation Antigens
VPLDCVLYRY

SEQ ID NO: 232 - protein, 9 aa, Artificial Sequence
  Differentiation Antigens
LPHSSSHWL

SEQ ID NO: 233 - protein, 9 aa, Artificial Sequence
  Differentiation Antigens
LPHSSSHWL

SEQ ID NO: 234 - protein, 8 aa, Artificial Sequence
  Differentiation Antigens
SNDGPTLI

SEQ ID NO: 235 - protein, 15 aa, Artificial Sequence
  Differentiation Antigens
GRAMLGTHTMEVTVY

SEQ ID NO: 236 - protein, 16 aa, Artificial Sequence
  Differentiation Antigens
WNRQLYPEWTEAQRLD

SEQ ID NO: 237 - protein, 18 aa, Artificial Sequence
  Differentiation Antigens
TTEWVETTARELPIPEPE

SEQ ID NO: 238 - protein, 17 aa, Artificial Sequence
  Differentiation Antigens
TGRAMLGTHTMEVTVYH

SEQ ID NO: 239 - protein, 15 aa, Artificial Sequence
  Differentiation Antigens
GRAMLGTHTMEVTVY

SEQ ID NO: 240 - protein, 9 aa, Artificial Sequence
  Differentiation Antigens
PLLENVISK

SEQ ID NO: 241 - protein, 11 aa, Artificial Sequence
  Differentiation Antigens
AEEAAGIGILT

SEQ ID NO: 242 - protein, 10 aa, Artificial Sequence
  Differentiation Antigens
AEEAAGIGIL

SEQ ID NO: 243 - protein, 11 aa, Artificial Sequence
  Differentiation Antigens
RNGYRALMDKS

SEQ ID NO: 244 - protein, 10 aa, Artificial Sequence
  Differentiation Antigens
EAAGIGILTV

SEQ ID NO: 245 - protein, 9 aa, Artificial Sequence
  Differentiation Antigens
AAGIGILTV

SEQ ID NO: 246 - protein, 30 aa, Artificial Sequence
  Differentiation Antigens
YTTAEEAAGIGILTVILGVLLLIGCWYCRR

SEQ ID NO: 247 - protein, 10 aa, Artificial Sequence
  Differentiation Antigens
EAAGIGILTV

SEQ ID NO: 248 - protein, 9 aa, Artificial Sequence
  Differentiation Antigens
ILTVILGVL

SEQ ID NO: 249 - protein, 12 aa, Artificial Sequence
  Differentiation Antigens
EEAAGIGILTVI

SEQ ID NO: 250 - protein, 14 aa, Artificial Sequence
  Differentiation Antigens
AAGIGILTVILGVL

SEQ ID NO: 251 - protein, 13 aa, Artificial Sequence
  Differentiation Antigens
APPAYEKLPSAEQ

SEQ ID NO: 252 - protein, 20 aa, Artificial Sequence
  Differentiation Antigens
MPREDAHFIYGYPKKGHGHS

SEQ ID NO: 253 - protein, 23 aa, Artificial Sequence
  Differentiation Antigens
RNGYRALMDKSLHVGTQCALTRR

SEQ ID NO: 254 - protein, 12 aa, Artificial Sequence
  Differentiation Antigens
EEAAGIGILTVI

SEQ ID NO: 255 - protein, 20 aa, Artificial Sequence
  Differentiation Antigens
KNCEPVVPNAPPAYEKLSAE

SEQ ID NO: 256 - protein, 9 aa, Artificial Sequence
  Differentiation Antigens
SLSKILDTV

SEQ ID NO: 257 - protein, 9 aa, Artificial Sequence
  Differentiation Antigens
LYSACFWWL

SEQ ID NO: 258 - protein, 9 aa, Artificial Sequence
  Differentiation Antigens
FLFLLFFWL

SEQ ID NO: 259 - protein, 9 aa, Artificial Sequence
  Differentiation Antigens
TLMSAMTNL

SEQ ID NO: 260 - protein, 9 aa, Artificial Sequence
  Differentiation Antigens
ALDVYNGLL

SEQ ID NO: 261 - protein, 10 aa, Artificial Sequence
  Differentiation Antigens
FLTPKKLQCV

SEQ ID NO: 262 - protein, 10 aa, Artificial Sequence
  Differentiation Antigens
VISNDVCAQV

SEQ ID NO: 263 - protein, 9 aa, Artificial Sequence
  Differentiation Antigens
VLHWDPETV

SEQ ID NO: 264 - protein, 9 aa, Artificial Sequence
  Differentiation Antigens
MSLQRQFLR

SEQ ID NO: 265 - protein, 15 aa, Artificial Sequence
  Differentiation Antigens
SQWRVVCDSLEDYDT

SEQ ID NO: 266 - protein, 10 aa, Artificial Sequence
  Differentiation Antigens
SLPYWNFATG

SEQ ID NO: 267 - protein, 21 aa, Artificial Sequence
  Differentiation Antigens
ISPNSVFSQWRVVCDSLEDYD

SEQ ID NO: 268 - protein, 9 aa, Artificial Sequence
  Differentiation Antigens
SVYDFFVWL

SEQ ID NO: 269 - protein, 9 aa, Artificial Sequence
  Differentiation Antigens
TLDSQVMSL

SEQ ID NO: 270 - protein, 9 aa, Artificial Sequence
  Differentiation Antigens
LLGPGRPYR

SEQ ID NO: 271 - protein, 9 aa, Artificial Sequence
  Differentiation Antigens
ANDPIFVVL

SEQ ID NO: 272 - protein, 15 aa, Artificial Sequence
  Differentiation Antigens
QCTEVRADTRPWSGP

SEQ ID NO: 273 - protein, 10 aa, Artificial Sequence
  Differentiation Antigens
ALPYWNFATG

SEQ ID NO: 274 - protein, 9 aa, Artificial Sequence
  Differentiation Antigens
KCDICTDEY

SEQ ID NO: 275 - protein, 11 aa, Artificial Sequence
  Differentiation Antigens
SSDYVIPIGTY

SEQ ID NO: 276 - protein, 9 aa, Artificial Sequence
  Differentiation Antigens
MLLAVLYCL

SEQ ID NO: 277 - protein, 10 aa, Artificial Sequence
  Differentiation Antigens
CLLWSFQTSA

SEQ ID NO: 278 - protein, 9 aa, Artificial Sequence
  Differentiation Antigens
YMDGTMSQV

SEQ ID NO: 279 - protein, 9 aa, Artificial Sequence
  Differentiation Antigens
AFLPWHRLF

SEQ ID NO: 280 - protein, 11 aa, Artificial Sequence
  Differentiation Antigens
IYMDGTADFSF

SEQ ID NO: 281 - protein, 12 aa, Artificial Sequence
  Differentiation Antigens
TPRLPSSADVEF

SEQ ID NO: 282 - protein, 9 aa, Artificial Sequence
  Differentiation Antigens
LPSSADVEF

SEQ ID NO: 283 - protein, 10 aa, Artificial Sequence
  Differentiation Antigens
LHHAFVDSIF

SEQ ID NO: 284 - protein, 15 aa, Artificial Sequence
  Differentiation Antigens
QNILLSNAPLGPQFP

SEQ ID NO: 285 - protein, 13 aa, Artificial Sequence
  Differentiation Antigens
SYLQDSDPDSFQD

SEQ ID NO: 286 - protein, 9 aa, Artificial Sequence
  Differentiation Antigens
QCSGNFMGF

SEQ ID NO: 287 - protein, 21 aa, Artificial Sequence [sequence omitted]

SEQ ID NO: 288 - protein, 9 aa, Artificial Sequence
  Differentiation Antigens
SEIWRDIDF

SEQ ID NO: 289 - protein, 9 aa, Artificial Sequence
  Antigens Overexpressed in Tumors
SVASTITGV

SEQ ID NO: 290 - protein, 10 aa, Artificial Sequence
  Antigens Overexpressed in Tumors
RSDSGQQARY

SEQ ID NO: 291 - protein, 9 aa, Artificial Sequence
  Antigens Overexpressed in Tumors
LLYKLADLI

SEQ ID NO: 292 - protein, 10 aa, Artificial Sequence
  Antigens Overexpressed in Tumors
YLNDHLEPWI

SEQ ID NO: 293 - protein, 9 aa, Artificial Sequence
  Antigens Overexpressed in Tumors
CQWGRLWQL

SEQ ID NO: 294 - protein, 10 aa, Artificial Sequence
  Antigens Overexpressed in Tumors
VLLQAGSLHA

SEQ ID NO: 295 - protein, 9 aa, Artificial Sequence
  Antigens Overexpressed in Tumors
KFLDALISL

SEQ ID NO: 296 - protein, 9 aa, Artificial Sequence
  Antigens Overexpressed in Tumors
KVHPVIWSL

SEQ ID NO: 297 - protein, 10 aa, Artificial Sequence
  Antigens Overexpressed in Tumors
LMLQNALTTM

SEQ ID NO: 298 - protein, 9 aa, Artificial Sequence
  Antigens Overexpressed in Tumors
LLGATCMFV

SEQ ID NO: 299 - protein, 15 aa, Artificial Sequence
  Antigens Overexpressed in Tumors
NPPSMVAAGSVVAAV

SEQ ID NO: 300 - protein, 10 aa, Artificial Sequence
  Antigens Overexpressed in Tumors
ALGGHPLLGV

SEQ ID NO: 301 - protein, 9 aa, Artificial Sequence
  Antigens Overexpressed in Tumors
TMNGSKSPV

SEQ ID NO: 302 - protein, 9 aa, Artificial Sequence
  Antigens Overexpressed in Tumors
RYQLDPKFI

SEQ ID NO: 303 - protein, 12 aa, Artificial Sequence
  Antigens Overexpressed in Tumors
DVTFNIICKKCG

SEQ ID NO: 304 - protein, 9 aa, Artificial Sequence
  Antigens Overexpressed in Tumors
FMVEDETVL

SEQ ID NO: 305 - protein, 9 aa, Artificial Sequence
  Antigens Overexpressed in Tumors
FINDEIFVE

SEQ ID NO: 306 - protein, 9 aa, Artificial Sequence
  Antigens Overexpressed in Tumors
KYDCFLHPF

SEQ ID NO: 307 - protein, 9 aa, Artificial Sequence
  Antigens Overexpressed in Tumors
KYVGIEREM

SEQ ID NO: 308 - protein, 9 aa, Artificial Sequence
  Antigens Overexpressed in Tumors
NTYASPRFK

SEQ ID NO: 309 - protein, 9 aa, Artificial Sequence
  Antigens Overexpressed in Tumors
FVGEFFTDV

SEQ ID NO: 310 - protein, 9 aa, Artificial Sequence
  Antigens Overexpressed in Tumors
EYILSLEEL

SEQ ID NO: 311 - protein, 9 aa, Artificial Sequence
  Antigens Overexpressed in Tumors
HLSTAFARV

SEQ ID NO: 312 - protein, 9 aa, Artificial Sequence
  Antigens Overexpressed in Tumors
KIFGSLAFL

SEQ ID NO: 313 - protein, 9 aa, Artificial Sequence
  Antigens Overexpressed in Tumors
IISAVVGIL

SEQ ID NO: 314 - protein, 9 aa, Artificial Sequence
  Antigens Overexpressed in Tumors
ALCRWGLLL

SEQ ID NO: 315 - protein, 9 aa, Artificial Sequence
  Antigens Overexpressed in Tumors
ILHNGAYSL

SEQ ID NO: 316 - protein, 9 aa, Artificial Sequence
  Antigens Overexpressed in Tumors
RLLQETELV

SEQ ID NO: 317 - protein, 9 aa, Artificial Sequence
  Antigens Overexpressed in Tumors
VVLGVVFGI

SEQ ID NO: 318 - protein, 10 aa, Artificial Sequence
  Antigens Overexpressed in Tumors
YMIMVKCWMI

SEQ ID NO: 319 - protein, 9 aa, Artificial Sequence
  Antigens Overexpressed in Tumors
HLYQGCQVV

SEQ ID NO: 320 - protein, 10 aa, Artificial Sequence
  Antigens Overexpressed in Tumors
YLVPQQGFFC

SEQ ID NO: 321 - protein, 9 aa, Artificial Sequence
  Antigens Overexpressed in Tumors
PLQPEQLQV

SEQ ID NO: 322 - protein, 9 aa, Artificial Sequence
  Antigens Overexpressed in Tumors
TLEEITGYL

SEQ ID NO: 323 - protein, 9 aa, Artificial Sequence
  Antigens Overexpressed in Tumors
ALIHHNTHL

SEQ ID NO: 324 - protein, 9 aa, Artificial Sequence
  Antigens Overexpressed in Tumors
PLTSIISAV

SEQ ID NO: 325 - protein, 9 aa, Artificial Sequence
  Antigens Overexpressed in Tumors
VLRENTSPK

SEQ ID NO: 326 - protein, 9 aa, Artificial Sequence
  Antigens Overexpressed in Tumors
TYLPTNASL

SEQ ID NO: 327 - protein, 9 aa, Artificial Sequence
  Antigens Overexpressed in Tumors
ALLEIASCL

SEQ ID NO: 328 - protein, 9 aa, Artificial Sequence
  Antigens Overexpressed in Tumors
NLSSAEVVV

SEQ ID NO: 329 - protein, 9 aa, Artificial Sequence
  Antigens Overexpressed in Tumors
RLLVPTQFV

SEQ ID NO: 330 - protein, 9 aa, Artificial Sequence
  Antigens Overexpressed in Tumors
WLPFGFILI

SEQ ID NO: 331 - protein, 9 aa, Artificial Sequence
  Antigens Overexpressed in Tumors
SPRWWPTCL

SEQ ID NO: 332 - protein, 9 aa, Artificial Sequence
  Antigens Overexpressed in Tumors
GVALQTMKQ

SEQ ID NO: 333 - protein, 9 aa, Artificial Sequence
  Antigens Overexpressed in Tumors
FMNKFIYEI

SEQ ID NO: 334 - protein, 10 aa, Artificial Sequence
  Antigens Overexpressed in Tumors
QLAVSVILRV

SEQ ID NO: 335 - protein, 9 aa, Artificial Sequence
  Antigens Overexpressed in Tumors
FLGYLILGV

SEQ ID NO: 336 - protein, 15 aa, Artificial Sequence
  Antigens Overexpressed in Tumors
SVSESDTIRSISIAS

SEQ ID NO: 337 - protein, 15 aa, Artificial Sequence
  Antigens Overexpressed in Tumors
LLANGRMPTVLQCVN

SEQ ID NO: 338 - protein, 15 aa, Artificial Sequence
  Antigens Overexpressed in Tumors
RMPTVLQCVNVSVVS

SEQ ID NO: 339 - protein, 9 aa, Artificial Sequence
  Antigens Overexpressed in Tumors
LLSDDDVVV

SEQ ID NO: 340 - protein, 10 aa, Artificial Sequence
  Antigens Overexpressed in Tumors
AQPDTAPLPV

SEQ ID NO: 341 - protein, 9 aa, Artificial Sequence
  Antigens Overexpressed in Tumors
CIAEQYHTV

SEQ ID NO: 342 - protein, 10 aa, Artificial Sequence
  Antigens Overexpressed in Tumors
FLPEFGISSA

SEQ ID NO: 343 - protein, 14 aa, Artificial Sequence
  Antigens Overexpressed in Tumors
LPAVVGLSPGEQEY

SEQ ID NO: 344 - protein, 16 aa, Artificial Sequence
  Antigens Overexpressed in Tumors
VGQDVSVLFRVTGALQ

SEQ ID NO: 345 - protein, 8 aa, Artificial Sequence
  Antigens Overexpressed in Tumors
VLFYLGQY

SEQ ID NO: 346 - protein, 9 aa, Artificial Sequence
  Antigens Overexpressed in Tumors
TLNDECWPA

SEQ ID NO: 347 - protein, 11 aa, Artificial Sequence
  Antigens Overexpressed in Tumors
FGRLQGISPKI

SEQ ID NO: 348 - protein, 14 aa, Artificial Sequence
  Antigens Overexpressed in Tumors
CPPWHPSERISSTL

SEQ ID NO: 349 - protein, 9 aa, Artificial Sequence
  Antigens Overexpressed in Tumors
GLPPDVQRV

SEQ ID NO: 350 - protein, 12 aa, Artificial Sequence
  Antigens Overexpressed in Tumors
SLFPNSPKWTSK

SEQ ID NO: 351 - protein, 9 aa, Artificial Sequence
  Antigens Overexpressed in Tumors
STAPPVHNV

SEQ ID NO: 352 - protein, 9 aa, Artificial Sequence
  Antigens Overexpressed in Tumors
LLLLTVLTV

SEQ ID NO: 353 - protein, 12 aa, Artificial Sequence
  Antigens Overexpressed in Tumors
PGSTAPPAHGVT

SEQ ID NO: 354 - protein, 9 aa, Artificial Sequence
  Antigens Overexpressed in Tumors
TCQPTCRSL

SEQ ID NO: 355 - protein, 9 aa, Artificial Sequence
  Antigens Overexpressed in Tumors
LLGRNSFEV

SEQ ID NO: 356 - protein, 9 aa, Artificial Sequence
  Antigens Overexpressed in Tumors
SQKTYQGSY

SEQ ID NO: 357 - protein, 13 aa, Artificial Sequence
  Antigens Overexpressed in Tumors
PGTRVRAMAIYKQ

SEQ ID NO: 358 - protein, 9 aa, Artificial Sequence
  Antigens Overexpressed in Tumors
RMPEAAPPV

SEQ ID NO: 359 - protein, 12 aa, Artificial Sequence
  Antigens Overexpressed in Tumors
HLIRVEGNLRVE

SEQ ID NO: 360 - protein, 9 aa, Artificial Sequence
  Antigens Overexpressed in Tumors
TLPGYPPHV

SEQ ID NO: 361 - protein, 12 aa, Artificial Sequence
  Antigens Overexpressed in Tumors
CTACRWKKACQR

SEQ ID NO: 362 - protein, 9 aa, Artificial Sequence
  Antigens Overexpressed in Tumors
VLDGLDVLL

SEQ ID NO: 363 - protein, 10 aa, Artificial Sequence
  Antigens Overexpressed in Tumors
SLYSFPEPEA

SEQ ID NO: 364 - protein, 10 aa, Artificial Sequence
  Antigens Overexpressed in Tumors
ALYVDSLFFL

SEQ ID NO: 365 - protein, 9 aa, Artificial Sequence
  Antigens Overexpressed in Tumors
SLLQHLIGL

SEQ ID NO: 366 - protein, 9 aa, Artificial Sequence
  Antigens Overexpressed in Tumors
NYARTEDFF

SEQ ID NO: 367 - protein, 9 aa, Artificial Sequence
  Antigens Overexpressed in Tumors
LKLSGVVRL

SEQ ID NO: 368 - protein, 10 aa, Artificial Sequence
  Antigens Overexpressed in Tumors
PLPPARNGGL

SEQ ID NO: 369 - protein, 10 aa, Artificial Sequence
  Antigens Overexpressed in Tumors
SPSSNRIRNT

SEQ ID NO: 370 - protein, 9 aa, Artificial Sequence
  Antigens Overexpressed in Tumors
LAALPHSCL

SEQ ID NO: 371 - protein, 10 aa, Artificial Sequence
  Antigens Overexpressed in Tumors
GLASFKSFLK

SEQ ID NO: 372 - protein, 10 aa, Artificial Sequence
  Antigens Overexpressed in Tumors
RAGLQVRKNK

SEQ ID NO: 373 - protein, 10 aa, Artificial Sequence
  Antigens Overexpressed in Tumors
ALWPWLLMAT

SEQ ID NO: 374 - protein, 9 aa, Artificial Sequence
  Antigens Overexpressed in Tumors
ALWPWLLMA

SEQ ID NO: 375 - protein, 9 aa, Artificial Sequence
  Antigens Overexpressed in Tumors
NSQPVWLCL

SEQ ID NO: 376 - protein, 9 aa, Artificial Sequence
  Antigens Overexpressed in Tumors
LPRWPPPQL

SEQ ID NO: 377 - protein, 9 aa, Artificial Sequence
  Antigens Overexpressed in Tumors
KMDAEHPEL

SEQ ID NO: 378 - protein, 9 aa, Artificial Sequence
  Antigens Overexpressed in Tumors
AWISKPPGV

SEQ ID NO: 379 - protein, 10 aa, Artificial Sequence
  Antigens Overexpressed in Tumors
SAWISKPPGV

SEQ ID NO: 380 - protein, 9 aa, Artificial Sequence
  Antigens Overexpressed in Tumors
MIAVFLPIV

SEQ ID NO: 381 - protein, 15 aa, Artificial Sequence
  Antigens Overexpressed in Tumors
HQQYFYKIPILVINK

SEQ ID NO: 382 - protein, 10 aa, Artificial Sequence
  Antigens Overexpressed in Tumors
ELTLGEFLKL

SEQ ID NO: 383 - protein, 15 aa, Artificial Sequence
  Antigens Overexpressed in Tumors
TLGEFLKLDRERAKN

SEQ ID NO: 384 - protein, 9 aa, Artificial Sequence
  Antigens Overexpressed in Tumors
ILAKFLHWL

SEQ ID NO: 385 - protein, 9 aa, Artificial Sequence
  Antigens Overexpressed in Tumors
RLVDDFLLV

SEQ ID NO: 386 - protein, 15 aa, Artificial Sequence
  Antigens Overexpressed in Tumors
LTDLQPYMRQFVAHL

SEQ ID NO: 387 - protein, 15 aa, Artificial Sequence
  Antigens Overexpressed in Tumors
RPGLLGASVLGLDDI

SEQ ID NO: 388 - protein, 9 aa, Artificial Sequence
  Antigens Overexpressed in Tumors
SRFGGAVVR

SEQ ID NO: 389 - protein, 11 aa, Artificial Sequence
  Antigens Overexpressed in Tumors
TSEKRPFMCAY

SEQ ID NO: 390 - protein, 9 aa, Artificial Sequence
  Antigens Overexpressed in Tumors
CMTWNQMNL

SEQ ID NO: 391 - protein, 11 aa, Artificial Sequence
  Antigens Overexpressed in Tumors
LSHLQMHSRKH

SEQ ID NO: 392 - protein, 16 aa, Artificial Sequence
  Antigens Overexpressed in Tumors
KRYFKLSHLQMHSRKH